(19) United States
(12) Patent Application Publication
(10) Pub. No.: US 2024/0307183 A1
(43) Pub. Date: Sep. 19, 2024

(54) SURGICAL ACCESS LOCATION

(71) Applicant: EDWARDS LIFESCIENCES CORPORATION, Irvine, CA (US)

(72) Inventors: Felino V. Cortez, JR., Bowie, MD (US); Luke Anthony Zanetti, Parkton, MD (US)

(21) Appl. No.: 18/679,336

(22) Filed: May 30, 2024

Related U.S. Application Data
(63) Continuation of application No. PCT/US2022/050957, filed on Nov. 23, 2022.
(60) Provisional application No. 63/264,731, filed on Dec. 1, 2021.

(51) Int. Cl. A61F 2/24

(52) U.S. Cl. CPC A61F 2/2457; A61F 2/2466

(57) ABSTRACT

A puncture locator device for use in locating a desired tissue puncture site and executing a puncture therethrough includes a handle, a tissue depressor form, and a needle channel that passes through at least a portion of the tissue depressor form.

### CROSS REFERENCE TO RELATED APPLICATION

This application is a continuation of International Patent Application No. PCT/US22/50957, filed on Nov. 23, 2022, which claims the benefit of U.S. Patent Application No. 63/264,731, filed on Dec. 1, 2021, the entire disclosures all of which are incorporated by reference for all purposes.

### BACKGROUND

The present disclosure generally relates to the field of medical procedures and devices. Various medical procedures involve accessing internal anatomy of a patient through biological tissue. The efficacy of such procedures can be affected at least in part by the particular access location and/or path utilized for tissue traversal.

### SUMMARY

Described herein are one or more methods and/or devices to facilitate puncture site and/or orientation location and execution. In some implementations, the present disclosure relates to a puncture locator device comprising a handle, a tissue depressor form, and a needle channel that passes through at least a portion of the tissue depressor form.

For purposes of summarizing the disclosure, certain aspects, advantages and novel features have been described. It is to be understood that not necessarily all such advantages may be achieved in accordance with any particular embodiment. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates an example representation of a human heart having a leaflet anchor deployed therein in accordance with one or more examples.

FIG. 2 is a perspective view of a tissue anchor delivery device in accordance with one or more examples.

FIGS. 3-1, 3-2, 3-3, 3-4, and 3-5 provide a flow diagram illustrating a process for implanting a leaflet anchor in accordance with one or more examples.

FIGS. 4-1, 4-2, 4-3, 4-4, and 4-5 provide images of cardiac anatomy and certain devices/systems corresponding to operations of the process of FIGS. 3-1, 3-2, 3-3, 3-4, and 3-5 in accordance with one or more examples.

FIG. 5 shows a cutaway view of a deployed leaflet anchor in a heart in accordance with one or more examples.

FIG. 6 shows a cutaway view of a deployed leaflet anchor in a heart in accordance with one or more examples.

FIG. 7-1 illustrates a view of an atrial/distal side of a valve having two tissue anchors deployed therein according to one or more examples.

FIG. 7-2 illustrates a view of an atrial/distal side of a valve having six tissue anchors deployed therein according to one or more examples.

FIGS. 8A-8G provide views of a puncture locator device in accordance with one or more examples.

FIGS. 9A-9E provide views of a puncture locator device in accordance with one or more examples.

FIGS. 10A and 10B show a puncture locator device pressed against a heart wall in accordance with one or more examples.

FIG. 11 shows a puncture locator device placed against a heart wall with an orientation indicator of the device oriented in a direction of a target anatomy in accordance with one or more examples.

FIGS. 12-1, 12-2, 12-3, and 12-4 provide a flow diagram illustrating a process for puncturing a tissue wall using a puncture locator device in accordance with one or more examples.

FIGS. 13-1, 13-2, 13-3, and 13-4 provide images of cardiac anatomy and certain devices/systems corresponding to operations of the process of FIGS. 12-1, 12-2, 12-3, and 12-4 in accordance with one or more examples.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular embodiment. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Certain standard anatomical terms of location are used herein to refer to certain device components/features and to the anatomy of animals, and namely humans, with respect to the preferred examples. Although certain spatially relative terms, such as “proximal,” “distal,” “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

The present disclosure relates to systems, devices, and methods for puncturing a biological tissue wall and/or for otherwise forming an access channel/path for accessing an anatomical chamber, lumen, or area through such access channel/path. Such through-tissue access may be implemented for certain tissue anchor deployment procedures, such as for deploying tissue/leaflet anchors in heart valve leaflets to tether the same to a heart wall, such as to serve as artificial chordae tendineae.

Devices and processes disclosed herein may be implemented in patients suffering from mitral regurgitation caused by, for example, mid-segment leaflet prolapse as a result of degenerative mitral valve disease. As referenced above, devices disclosed herein may be suited for use in connection with procedures for delivering and anchoring artificial chordae devices/components (e.g., ePTFE cords) to a prolapse mitral valve leaflet in a beating-heart procedure. Access to the target leaflet may be made through a puncture/access channel in the ventricular heart wall, wherein the location of such puncture may be located and/or facilitated through the use of a puncture locator device including a needle channel through which a needle shaft may be inserted to puncture the heart wall at a desired angle and/or to a desired depth of puncture.

Puncture locator devices in accordance with aspects of the present disclosure may have associated therewith certain components, described in detail below, such as a manually manipulable handle, a tissue depressor form/feature, and/or a stabilizer structure for stabilizing the device against the exterior (e.g., epicardium/pericardium) wall of the heart. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

Certain examples are disclosed herein in the context of cardiac implants and procedures. However, although certain principles disclosed herein are particularly applicable to the anatomy of the heart, it should be understood that puncture locator devices and puncture/access procedures in accordance with the present disclosure may be implanted in, or configured for implantation in, any suitable or desirable anatomy.

### Cardiac Physiology

The following includes a general description of human cardiac anatomy that is relevant to certain inventive features and examples disclosed herein and is included to provide context for certain aspects of the present disclosure. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

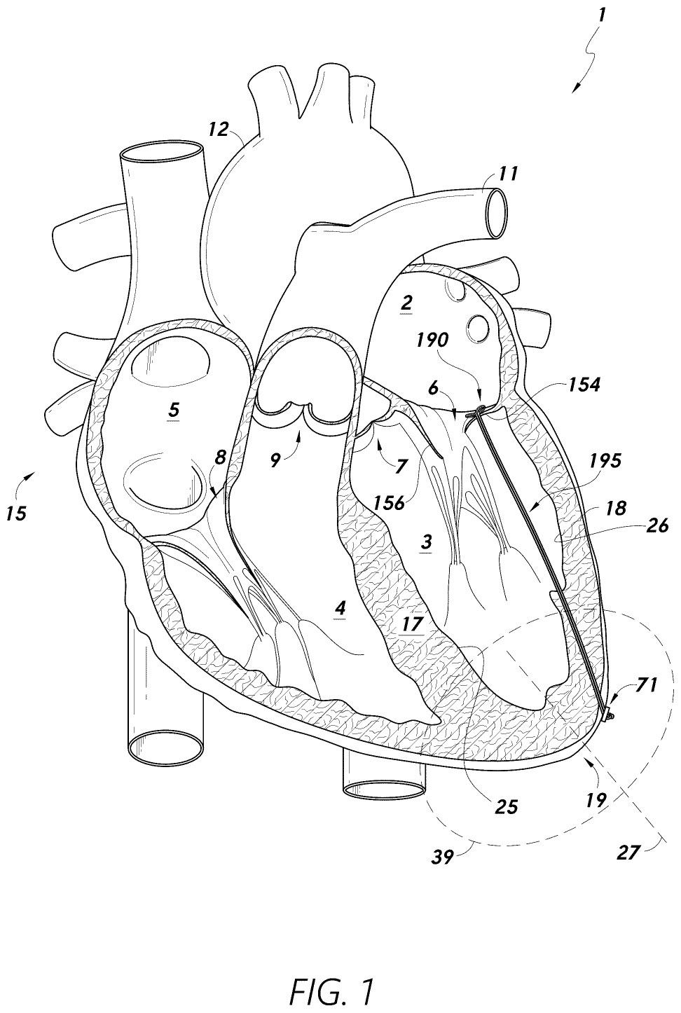

FIG. 1 illustrates an example representation of a heart 1 having various features relevant to certain aspects of the present inventive disclosure. The heart 1 includes four chambers, namely the left ventricle 3, the left atrium 2, the right ventricle 4, and the right atrium 5. A wall of muscle 17, referred to as the septum, separates the left 2 and right 5 atria and the left 3 and right 4 ventricles. The inferior tip 19 of the heart 1 is referred to as the apex and is generally located on the midclavicular line, in the fifth intercostal space. The apex 19 can be considered part of the greater apical region 39.

The left ventricle 3 is the primary pumping chamber of the heart 1. A healthy left ventricle is generally conical or apical in shape in that it is longer (along a longitudinal axis extending in a direction from the aortic valve 7 to the apex 19) than it is wide (along a transverse axis extending between opposing walls 25, 26 at the widest point of the left ventricle) and descends from a base 15 with a decreasing cross-sectional circumference to the point or apex 19. Generally, the apical region 39 of the heart is a bottom region of the heart that is within the left or right ventricular region but is distal to the mitral 6 and tricuspid 8 valves and toward the tip of the heart. More specifically, the apical region 39 may be considered to be within about 20 cm to the right or to the left of the median axis 27 of the heart 1.

The pumping of blood from the left ventricle is accomplished by a squeezing motion and a twisting or torsional motion. The squeezing motion occurs between the lateral wall 18 of the left ventricle and the septum 17. The twisting motion is a result of heart muscle fibers that extend in a circular or spiral direction around the heart. When these fibers contract, they produce a gradient of angular displacements of the myocardium from the apex 19 to the base 15 about the longitudinal axis of the heart. The resultant force vectors extend at angles from about 30-60 degrees to the flow of blood through the aortic valve 7. The contraction of the heart is manifested as a counterclockwise rotation of the apex 19 relative to the base 15, when viewed from the apex 19. A healthy heart can pump blood from the left ventricle in a very efficient manner due to the spiral contractility of the heart.

The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (e.g., systole) and open during ventricular expansion (e.g., diastole). The valves of the heart 1 further include the pulmonary valve 9, which separates the right ventricle 4 from the pulmonary artery 11 and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The atrioventricular (e.g., mitral and tricuspid) heart valves may comprise a collection of chordae tendineae (13, 16) and papillary muscles (10, 15) for securing the leaflets of the respective valves to promote and/or facilitate proper coaptation of the valve leaflets and prevent prolapse thereof. The papillary muscles, for example, may generally comprise finger-like projections from the ventricle wall. With respect to the tricuspid valve 8, the normal tricuspid valve may comprise three leaflets and three corresponding papillary muscles 10 (two shown in FIG. 1). The leaflets of the tricuspid valve may be referred to as the anterior, posterior and septal leaflets, respectively. The valve leaflets are connected to the papillary muscles 10 by the chordae tendineae 13, which are disposed in the right ventricle 4 along with the papillary muscles 10.

Surrounding the ventricles (3, 4) are a number of arteries (not shown) that supply oxygenated blood to the heart muscle and a number of veins that return the blood from the heart muscle. The coronary sinus (not shown) is a relatively large vein that extends generally around the upper portion of the left ventricle 3 and provides a return conduit for blood returning to the right atrium 5. The coronary sinus terminates at the coronary ostium (not shown) through which the blood enters the right atrium.

With respect to the mitral valve 6, a normal mitral valve may comprise two leaflets (anterior and posterior) and two corresponding papillary muscles 15. The papillary muscles 15 originate in the left ventricle wall and project into the left ventricle 3. Generally, the anterior leaflet may cover approximately two-thirds of the valve annulus. Although the anterior leaflet covers a greater portion of the annulus, the posterior leaflet may comprise a larger surface area in certain anatomies.

Various disease processes can impair the proper functioning of one or more of the valves of the heart. These disease processes include degenerative processes (e.g., Barlow's Disease, fibroelastic deficiency), inflammatory processes (e.g., rheumatic heart disease) and infectious processes (e.g., endocarditis). Additionally, damage to the ventricle from prior heart attacks (e.g., myocardial infarction secondary to coronary artery disease) or other heart diseases (e.g., cardiomyopathy) can distort the valve's geometry causing it to dysfunction. However, the vast majority of patients undergoing valve surgery, such as mitral valve surgery, suffer from a degenerative disease that causes a malfunction in one or more leaflets of the valve which results in prolapse and regurgitation.

The mitral valve 6 and tricuspid valve 8 can be divided into three parts: an annulus, leaflets, and a sub-valvular apparatus. The sub-valvular apparatus can be considered to include the papillary muscles 10, 15 and the chordae tendineae 13, 16, which can elongate and/or rupture. If a valve is functioning properly, when closed, the free margins or edges of the leaflets come together and form a tight junction, the arc of which, in the mitral valve, is known as the line, plane or area of coaptation. Normal mitral and tricuspid valves open when the ventricles relax allowing blood from the atrium to fill the decompressed ventricle. When the ventricle contracts, the chordae tendineae advantageously properly tether or position the valve leaflets such that the increase in pressure within the ventricle causes the valve to close, thereby preventing blood from leaking into the atrium and assuring that substantially all of the blood leaving the ventricle is ejected through the aortic valve 7 or pulmonic valve 9 and into the arteries of the body. Accordingly, proper function of the valves depends on a complex interplay between the annulus, leaflets, and sub-valvular apparatus. Lesions in any of these components can cause the valve to dysfunction and thereby lead to valve regurgitation.

Generally, there are three mechanisms by which a heart valve becomes regurgitant or incompetent; they include Carpentier's type I, type II and type III malfunctions. A Carpentier type I malfunction involves the dilation of the annulus such that normally functioning leaflets are distracted from each other and fail to form a tight seal (e.g., do not coapt properly). Included in a type I mechanism malfunction are perforations of the valve leaflets, as in endocarditis. A Carpentier's type II malfunction involves prolapse of one or both leaflets above the plane of coaptation. This is the most common cause of mitral regurgitation and is often caused by the stretching or rupturing of chordae tendineae normally connected to the leaflet. A Carpentier's type III malfunction involves restriction of the motion of one or more leaflets such that the leaflets are abnormally constrained below the level of the plane of the annulus. Leaflet restriction can be caused by rheumatic disease (IIIa) or dilation of the ventricle (IIIb).

One or more chambers in the heart 1 may be accessed in accordance with certain heart valve-repair procedures and/or other interventions. Access into a chamber in the heart may be made at any suitable site of entry. In some implementations, access is made to a chamber of the heart, such as a target ventricle (e.g., left ventricle) associated with a diseased heart valve, through the apical region 39. For example, access into the left ventricle 3 (e.g., to perform a mitral valve repair) may be gained by making a relatively small incision at the apical region 39, close to (or slightly skewed toward the left of) the median axis 27 of the heart. Access into the right ventricle 4 (e.g., to perform a tricuspid valve repair) may be gained by making a small incision into the apical region 39, close to or slightly skewed toward the right of the median axis 27 of the heart. Accordingly, the ventricle can be accessed directly via the apex, or via an off-apex location that is in the apical region 39 but slightly removed from the tip/apex, such as via lateral ventricular wall, a region between the apex and the base of a papillary muscle, or even directly at the base of a papillary muscle. In some implementations, the incision made to access the appropriate ventricle of the heart is no longer than about 1 mm to about 5 cm, from 2.5 mm to about 2.5 cm, or from about 5 mm to about 1 cm in length. When a percutaneous approach is sought, no incision into the apex region of the heart may be made, but rather access into the apical region 39 may be gained by direct needle puncture, for instance by an 18-gauge needle, through which an appropriate repair instrument can be advanced.

### Heart Valve Leaflet Tethering

Certain inventive features disclosed herein relate to certain heart valve repair systems and devices, and/or systems, process, and devices for repairing any other type of target organ tissue. In some implementations, a tissue anchor delivery device may be employed in repairing a mitral valve in patients suffering from degenerative mitral regurgitation or other condition. In some implementations, a transapical, off-pump repair procedure is implemented in which at least part (e.g., a shaft portion/assembly) of a valve repair system is inserted in the left ventricle and advanced to the surface of the diseased portion of a target mitral valve leaflet and used to deploy/implant a tissue anchor in the target leaflet. The tissue anchor (e.g., sutureform formed into a bulky knot) may advantageously be integrated or coupled with one or more artificial/synthetic cords serving a function similar to that of chordae tendineae. Such artificial cord(s) may comprise suture(s) and/or suture tail portions associated with a knot-type tissue anchor and may comprise any suitable or desirable material, such as expanded polytetrafluoroethylene (ePTFE) or the like. The term “suture” is used herein according to its broad and ordinary meaning and may refer to any elongate cord, strip, strand, line, tie, string, ribbon, strap, or portion thereof, or other type of material used in medical procedures. One having ordinary skill in the art will understand that a wire or other similar material may be used in place of a suture. Furthermore, in some contexts herein, the terms “cord,” “chord,” “chordae,” and “suture” may be used substantially interchangeably. In addition, use of the singular form of any of the suture-related terms listed above, including the terms “suture” and “cord,” may be used to refer to a single suture/cord, or to a portion thereof, or to a plurality of suture/cords, such as a pair of suture/cord tails emanating from a single anchor, knot, form, device, or other structure or assembly. Where a suture knot or anchor is deployed on a distal side of a tissue portion, and where two suture portions extend from the knot/anchor on a proximal side of the tissue, either or both of the suture portions may be referred to as a “suture” or a “cord,” regardless of whether both portions are part of a unitary suture or cord or are separate.

Processes for repairing a target organ tissue, such as repair of mitral valve leaflets to address mitral valve regurgitation, can include inserting a tissue anchor delivery device, such as a delivery device as described in PCT Application No. PCT/US2012/043761, (published as WO 2013/003228, and referred to herein as “the '761 PCT Application”) and/or in PCT Application No. PCT/US2016/055170 (published as WO 2017/059426 and referred to herein as “the '170 PCT Application”), the entire disclosures of which are incorporated herein by reference, into a body and extending a distal end of the delivery device to a proximal side of the target tissue (e.g., leaflet).

The '761 PCT Application and the '170 PCT Application describe in detail methods and devices for performing non-invasive procedures to repair a cardiac valve, such as a mitral valve. Such procedures include procedures to repair regurgitation that occurs when the leaflets of the mitral valve do not coapt properly at peak contraction pressures, resulting in an undesired backflow of blood from the ventricle into the atrium. As described in the '761 PCT Application and the '170 PCT Application, after the malfunctioning cardiac valve has been assessed and the source of the malfunction verified, a corrective procedure can be performed. Various procedures can be performed in accordance with the methods described therein to effectuate a cardiac valve repair, which may depend on the specific abnormality and the tissues involved.

With further reference to FIG. 1, FIG. 1 shows an example deployed leaflet/tissue anchor 190 deployed in a hear valve leaflet 154 and tethered to a heart/ventricle wall 18 via one or more sutures/suture tails 195 coupled to and/or associated with the anchor 190. The suture tails 195 coupled to the anchor 190 may be secured at the desired tension using a pledget 71 or other suture-fixing/locking device or mechanism on the outside of the heart wall 18 through which the suture tails 195 may run. A knot or other suture fixation mechanism or device may be implemented to hold the sutures at the desired tension and to the pledget 71. With the suture tail(s) 195 fixed to the ventricle wall 18, a portion of the suture tail(s) 195 disposed within the ventricle 3 may advantageously function as replacement leaflet cords (e.g., chordae tendineae) that are configured to tether the target leaflet 154 in a desired manner.

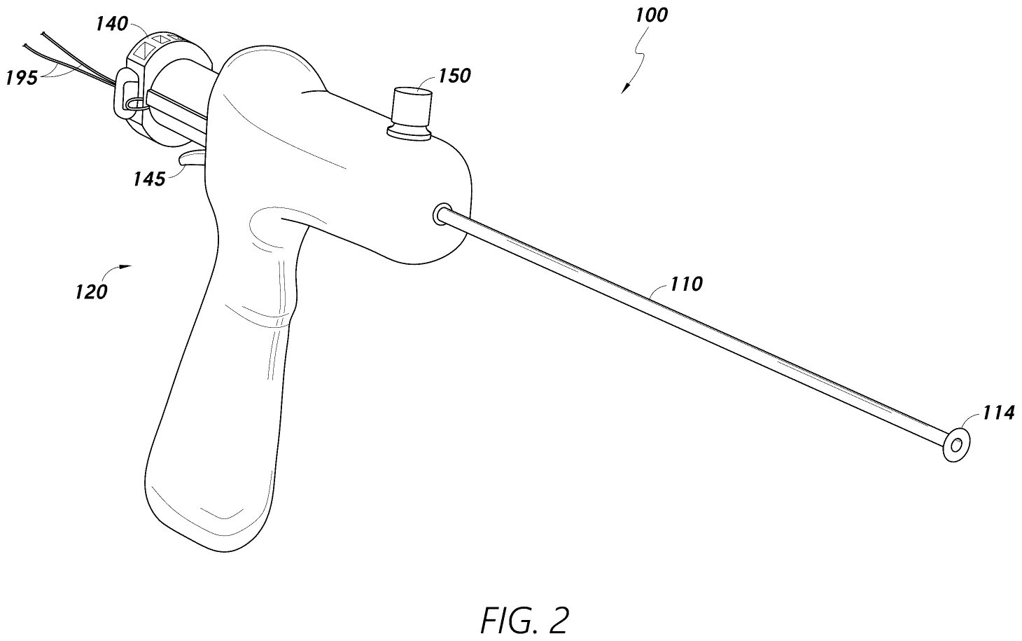

FIG. 2 is a perspective view of a tissue anchor delivery device in accordance with one or more examples. The tissue anchor delivery system 100 may be used to repair a heart valve, such as a mitral valve, and improve functionality thereof. For example, the tissue anchor delivery system 100 may be used to reduce the degree of mitral regurgitation in patients suffering from mitral regurgitation caused by, for example, midsegment prolapse of valve leaflets as a result of degenerative mitral valve disease. In order to repair such a valve, the tissue anchor delivery system 100 may be utilized to deliver and anchor tissue anchors, such as suture-knot-type tissue anchors, in a prolapsed valve leaflet. As described in detail below, such procedure may be implemented on a beating heart.

The delivery system 100 includes a rigid elongate tube 110 forming at least one internal working lumen. Although described in certain examples and/or contexts as comprising a rigid elongate tube, it should be understood that tubes, shafts, lumens, conduits, and the like disclosed herein may be either rigid, at least partially rigid, at least flexible, and/or at least partially flexible. Therefore, any such component described herein, whether or not referred to as rigid herein should be interpreted as possibly being at least partially flexible. In accordance with the present disclosure, the rigid elongate tube 110 may be referred to as a shaft for simplicity. Implementation of a valve-repair procedure utilizing the delivery system 100 can be performed in conjunction with certain imaging technology designed to provide visibility of the shaft 110 of the delivery system 100 according to a certain imaging modality, such as echo imaging. Generally, when performing a valve-repair procedure utilizing the tissue anchor delivery system 100, the operating physician may advantageously work in concert with an imaging technician, who may coordinate with the physician to facilitate successful execution of the valve-repair procedure.

In addition to the delivery shaft 110, the delivery system 100 may include a plunger feature 140, which may be used or actuated to manually deploy a pre-formed knot, such as a bulky knot as described in detail below. The tissue anchor delivery system 100 may further include a plunger lock mechanism 145, which may serve as a safety lock that locks the valve delivery system until ready for use or deployment of a leaflet anchor as described herein. The plunger 140 may have associated therewith a suture-release mechanism, which may be configured to lock in relative position a pair of suture tails 195 associated with a pre-formed knot anchor (not shown) to be deployed. For example, the suture portions 195 may be ePTFE sutures. The system 100 may further comprise a flush port 150, which may be used to de-air the lumen of the shaft 110. For example, heparinized saline flush, or the like, may be connected to the flush port 150 using a female Luer fitting to de-air the valve repair system 100. The term “lumen” is used herein according to its broad and ordinary meaning, and may refer to a physical structure forming a cavity, void, pathway, or other channel, such as an at least partially rigid elongate tubular structure, or may refer to a cavity, void, pathway, or other channel, itself, that occupies a space within an elongate structure (e.g., a tubular structure). Therefore, with respect to an elongate tubular structure, such as a shaft, tube, or the like, the term “lumen” may refer to the elongate tubular structure and/or to the channel or space within the elongate tubular structure.

The lumen of the shaft 110 may house a needle (not shown) that is wrapped at least in part with a pre-formed knot sutureform anchor, as described in detail herein. In some examples, the shaft 110 presents a relatively low profile. For example, the shaft 110 may have a diameter of approximately 3 mm or less (e.g., 9 Fr). The shaft 110 is associated with an atraumatic tip 114 feature. The atraumatic tip 114 can be an echogenic leaflet-positioner component, which may be used for deployment and/or positioning of the suture-type tissue anchor. The atraumatic tip 114, disposed at the distal end of the shaft 110, may be configured to have deployed therefrom a wrapped pre-formed suture knot (e.g., sutureform), as described herein.

The atraumatic tip 114 may be referred to as an “end effector.” In addition to a pre-formed knot sutureform and associated needle, the shaft 110 may house an elongated knot pusher tube (not shown; also referred to herein as a “pusher”), which may be actuated using the plunger 140 in some examples. As described in further detail below, the tip 114 provides a surface against which the target valve leaflet may be held in connection with deployment of a leaflet anchor.

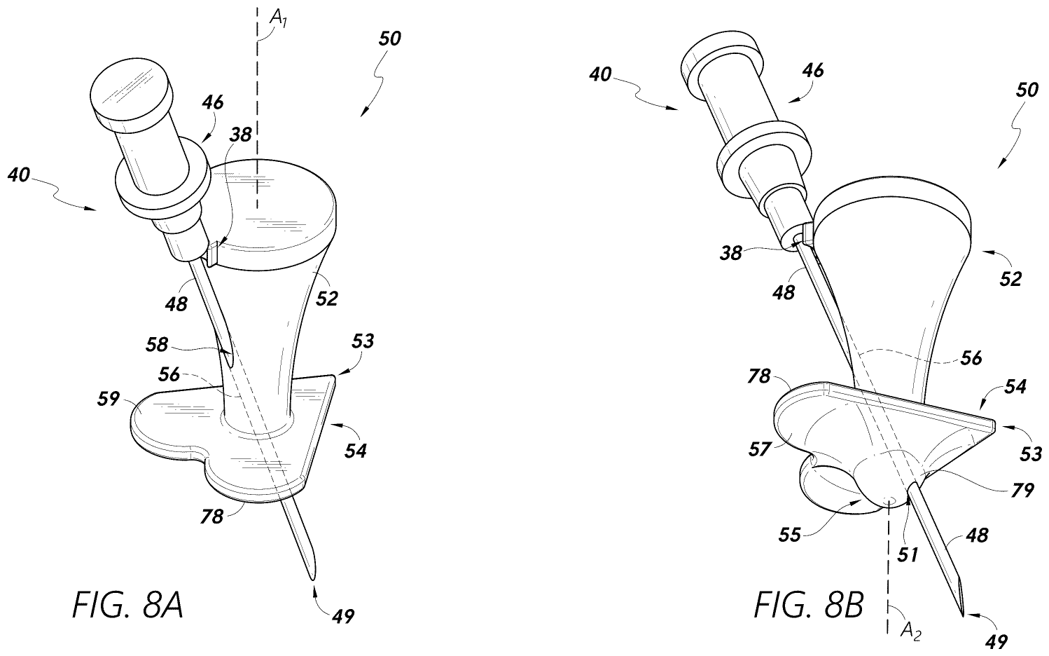

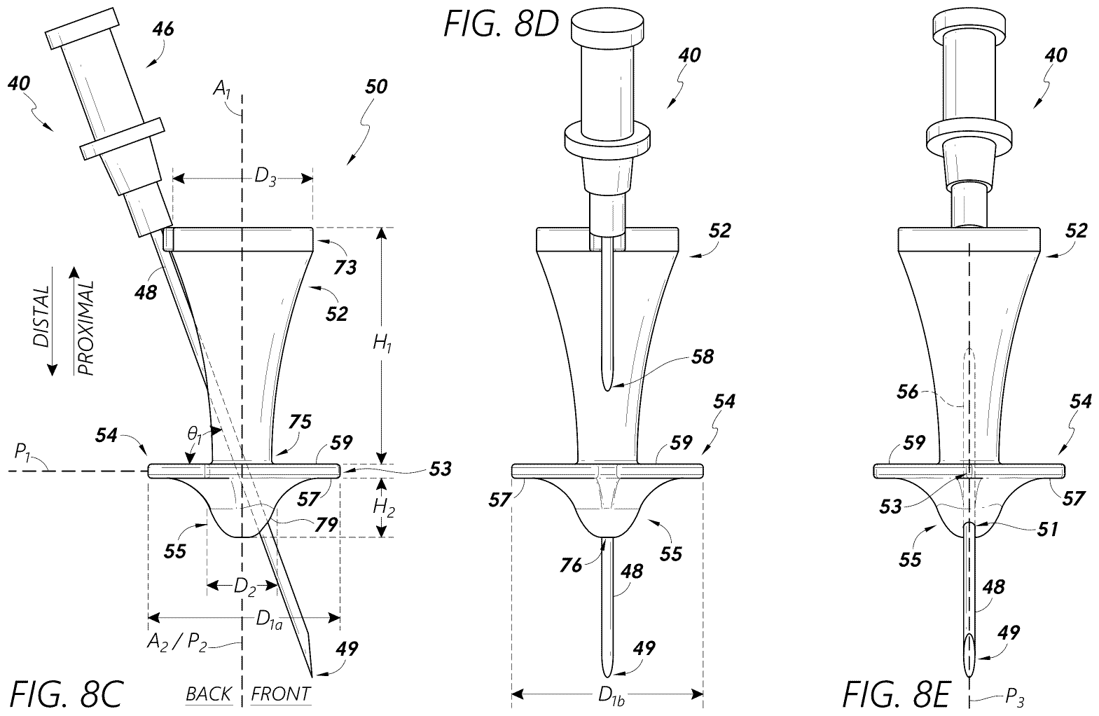

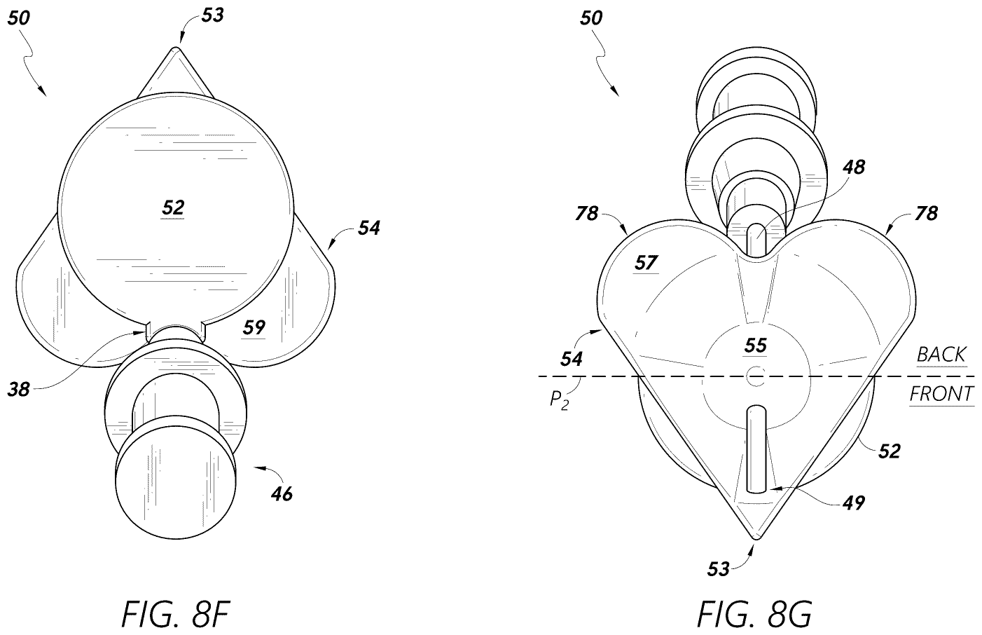

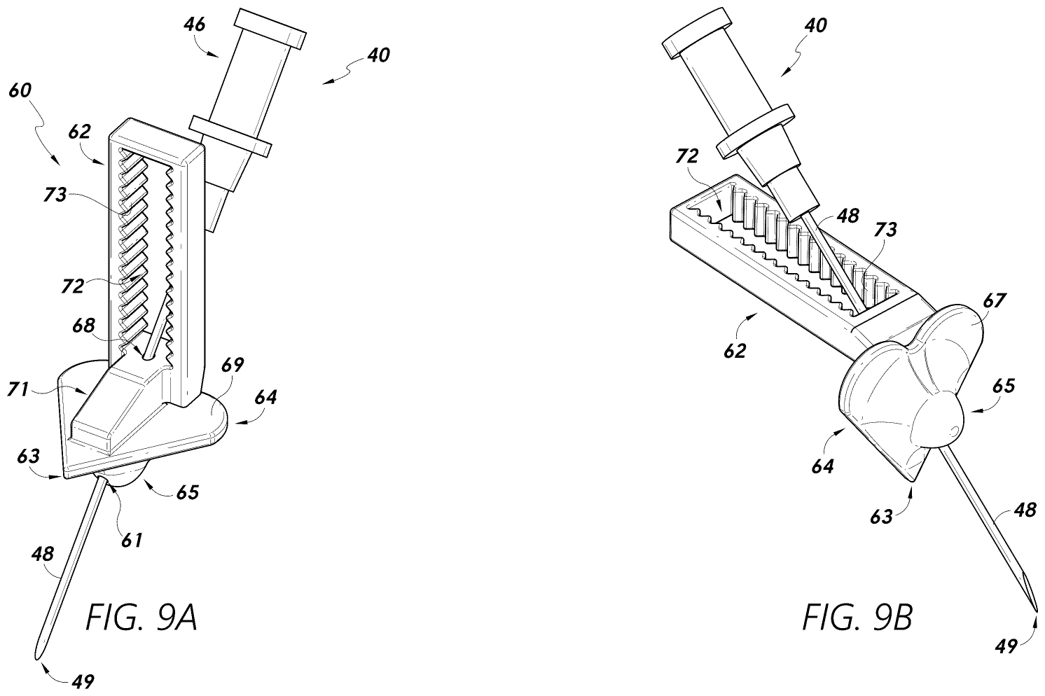

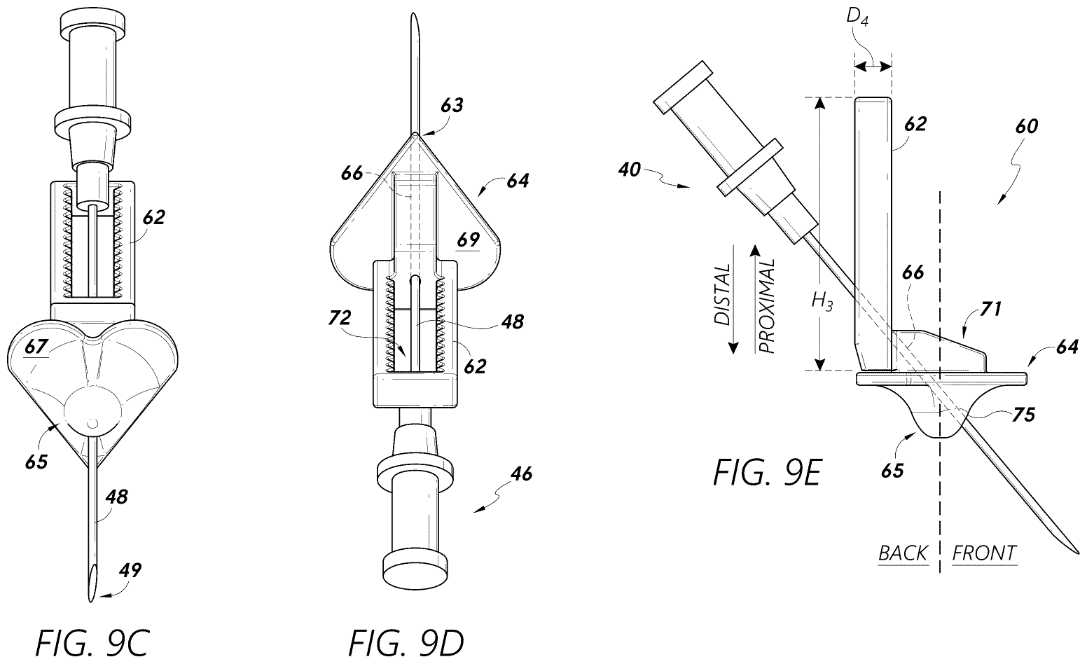

The delivery device 100 may be used to deliver a “bulky knot” type tissue anchor, as described in greater detail below. For example, the delivery device 100 may be utilized to deliver a tissue anchor (e.g., bulky knot) on a distal side of a mitral valve leaflet. The tip 114 (e.g., end effector), can be placed in contact with the ventricular side of a leaflet of a mitral valve. The tip 114 can be coupled to the distal end portion of the shaft 110, wherein the proximal end portion of the shaft 110 may be coupled to a handle portion 120 of the delivery device 100, as shown. Generally, the elongate pusher (not shown) may be movably disposed within a lumen of the shaft 110 and coupled to a pusher hub (not shown) that is movably disposed within the handle 120 and releasably coupled to the plunger 140. A needle (not shown) carrying a pre-formed tissue anchor sutureform can be movably disposed within a lumen of the pusher and coupled to a needle hub (not shown) that is also coupled to the plunger 140. The plunger 140 can be used to actuate or move the needle and the pusher during deployment of a distal anchor (see, e.g., FIGS. 8 and 9) and is movably disposed at least partially within the handle 120. For example, the handle 120 may define a lumen in which the plunger 140 can be moved. During operation, the pusher may also move within the lumen of the handle 120. The plunger lock 145 can be used to prevent the plunger 140 from moving within the handle 120 during storage and prior to performing a procedure to deploy a tissue anchor.

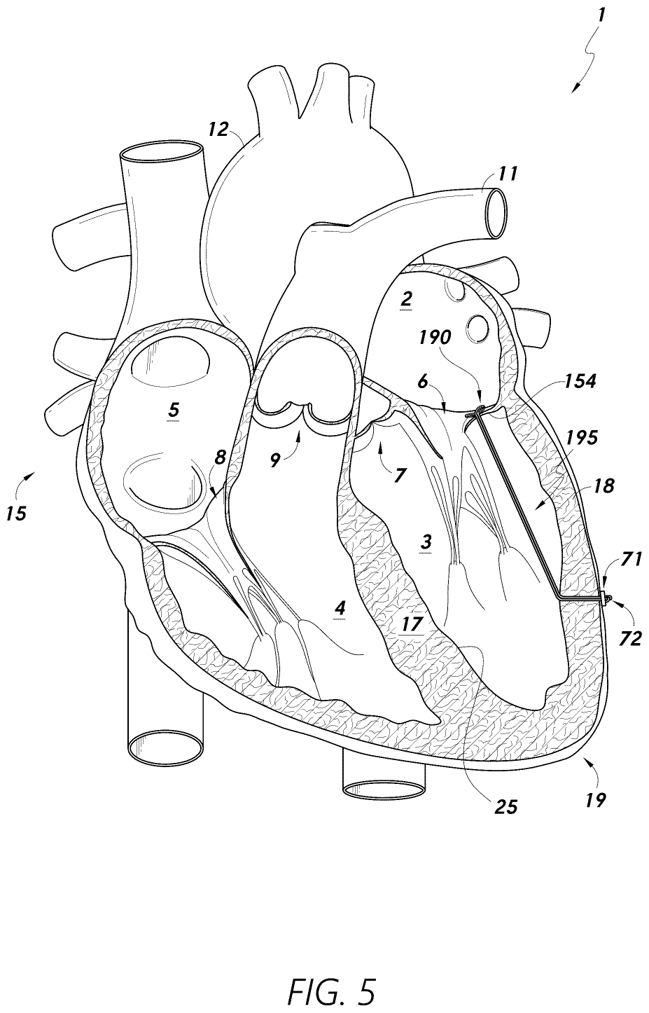

The needle may have the pre-formed knot disposed about a distal portion thereof while maintained in the shaft 110. For example, the pre-formed knot may be formed of one or more sutures configured in a coiled sutureform (see image 418 of FIG. 4-5) having a plurality of winds/turns around the needle over a portion of the needle that is associated with a longitudinal slot in the needle that runs from the distal end thereof. Although the term “sutureform” is used herein, it should be understood that such components/forms may comprise suture, wire, or any other elongate material wrapped or formed in a desired configuration. The coiled sutureform can be provided or shipped disposed around the needle. In some instances, two suture tails extend from the coiled sutureform. The suture tails 195 may extend through the lumen of the needle and/or through a passageway of the plunger 140 and may exit the plunger 140 at a proximal end portion thereof. The coiled sutureform may advantageously be configured to be formed into a suture-type tissue anchor (referred to herein as a “bulky knot”) in connection with an anchor-deployment procedure, as described in more detail below. The coiled sutureform can be configurable to a knot/deployed configuration by approximating opposite ends of the coiled portion thereof towards each other to form one or more loops.

The delivery device/system 100 can further include a suture/tether catch mechanism (not shown) coupled to the plunger 140 at a proximal end of the delivery device 100, which may be configured to releasably hold or secure a suture 195 extending through the delivery device 100 during delivery of a tissue anchor as described herein. The suture catch can be used to hold the suture 195 with a friction fit or with a clamping force and can have a lock that can be released after the tissue anchor has been deployed/formed into a bulky knot, as described herein.

As described herein, the anchor delivery device 100 can be used in beating heart mitral valve repair procedures. In some instances, the shaft 110 of the delivery device 100 can be configured to extend and contract with the beating of the heart. During systolic contraction, the median axis of the heart generally shortens. For example, with reference to FIG. 1, the distance from the apex 19 of the heart to the valve leaflets 152, 154 can vary by about 1 cm to about 2 cm with each heartbeat in some patients. In some instances, the length of the shaft 110 that protrudes from the handle 120 can change with the length of the median axis of the heart. That is, distal end of the shaft 110 can be configured to be floating such that the shaft can extend and retract with the beat of the heart so as to maintain contact with the target mitral valve leaflet.

Advancement of the delivery device 100 may be performed in conjunction with echo imaging, direct visualization (e.g., direct transblood visualization), and/or any other suitable remote visualization technique/modality. With respect to cardiac procedures, for example, the delivery device 100 may be advanced in conjunction with transesophageal (TEE) guidance and/or intracardiac echocardiography (ICE) guidance to facilitate and to direct the movement and proper positioning of the device for contacting the appropriate target cardiac region and/or target cardiac tissue (e.g., a valve leaflet, a valve annulus, or any other suitable cardiac tissue). Typical procedures that can be implemented using echo guidance are set forth in Suematsu, Y., J. Thorac. Cardiovasc. Surg. 2005; 130:1348-56 (“Suematsu”), the entire disclosure of which is incorporated herein by reference.

FIGS. 3-1, 3-2, 3-3, 3-4, and 3-5 provide a flow diagram illustrating a process 300 for implanting a leaflet anchor in accordance with one or more examples. FIGS. 4-1, 4-2, 4-3, 4-4, and 4-5 provide images of cardiac anatomy and certain devices/systems corresponding to operations of the process of FIGS. 3-1, 3-2, 3-3, 3-4, and 3-5 in accordance with one or more examples.

The process 300 may be implemented when a minimally invasive approach is determined to be advisable. Although not shown specifically in the flow diagram of FIGS. 3-1 through 3-5, the process may initially involve making one or more incisions proximate to the thoracic cavity to provide a surgical field of access. The total number and length of the incisions to be made depend on the number and types of the instruments to be used as well as the procedure(s) to be performed. The incision(s) may advantageously be made in such a manner as to be minimally invasive. As referred to herein, the term “minimally invasive” means in a manner by which an interior organ or tissue may be accessed with relatively little damage being done to the anatomical structure through which entry is sought. For example, a minimally invasive procedure may involve accessing a body cavity by a small incision of, for example, approximately 5 cm or less made in the skin of the body. The incision may be vertical, horizontal, or slightly curved. If the incision is located along one or more ribs, it may advantageously follow the outline of the rib. The opening may advantageously extend deep enough to allow access to the thoracic cavity between the ribs or under the sternum and is preferably set close to the rib cage and/or diaphragm, dependent on the entry point chosen.

In one example method, the heart may be accessed through one or more openings made by one or more small incision in a portion of the body proximal to the thoracic cavity, such as between one or more of the ribs of the rib cage of a patient, proximate to the xyphoid appendage, or via the abdomen and diaphragm. Access to the thoracic cavity may be sought to allow the insertion and use of one or more thorascopic instruments, while access to the abdomen may be sought to allow the insertion and use of one or more laparoscopic instruments. Insertion of one or more visualizing instruments may then be followed by transdiaphragmatic access to the heart. Additionally, access to the heart may be gained by direct puncture (e.g., via an appropriately sized needle, for instance an 18-gauge needle) of the heart from the xyphoid region. Accordingly, the one or more incisions should be made in such a manner as to provide an appropriate surgical field and access site to the heart in the least invasive manner possible. Access may also be achieved using percutaneous methods, further reducing the invasiveness of the procedure. See, e.g., “Full-Spectrum Cardiac Surgery Through a Minimal Incision Mini-Sternotomy (Lower Half) Technique,” Doty et al., Annals of Thoracic Surgery 1998; 65(2): 573-7 and “Transxiphoid Approach Without Median Sternotomy for the Repair of Atrial Septal Defects,” Barbero-Marcial et al., Annals of Thoracic Surgery 1998; 65(3): 771-4, the entire disclosures of each of which are incorporated herein by reference.

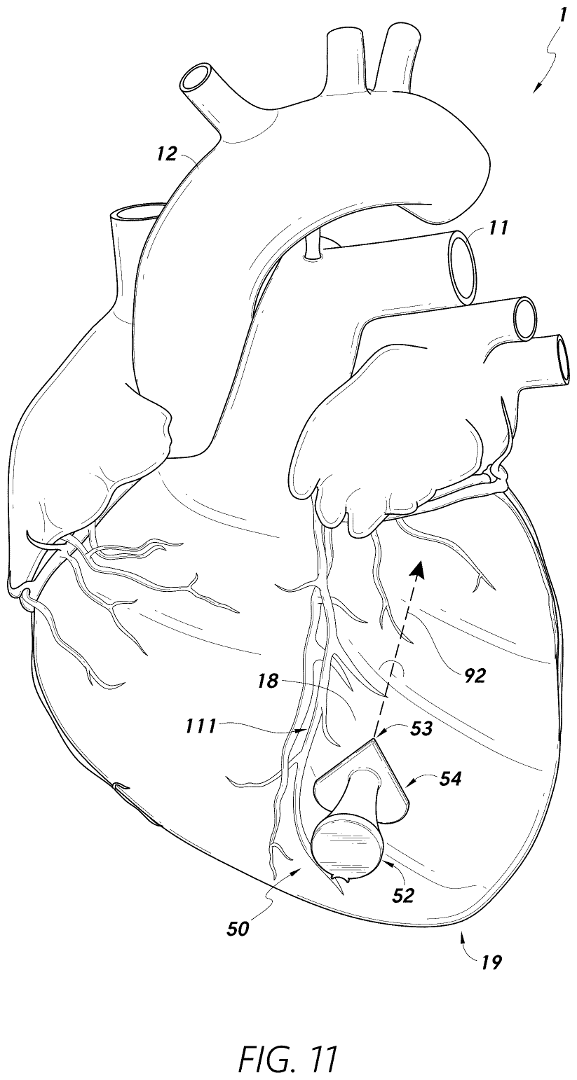

The process 300 involves, at block 302, identifying a site/location for introducer insertion through the wall 18 of a heart 1. For example, as shown in the image 402, the identification of the puncture/introducer insertion location may involve implementing a finger-poke test/action, wherein the surgeon may depress the heart wall 18 with his or her finger 109 at a proposed left ventricular access site, which may be approximately 2 to 4 cm basil from the true apex of the left ventricle, just lateral to the left anterior descending artery (LAD) 111 (see FIG. 11). However, as different fingers of a given individual and/or fingers of different individuals can vary in size, finger-poke processes may not provide consistency in results across procedures/individuals.

The finger's displacement of the ventricular wall 18 can be imaged with certain imaging technology (e.g., transesophageal echocardiography (TEE) imaging or other modality) in order to verify the optimal introducer site. For example, the depression of the heart wall 18 may visibly show in the imaging window. Generally, the optimal puncture/introducer path/site may be positioned between the papillary muscles 108 of the ventricle 3. Although described as using a finger poke test, certain implementations of the process 300 may advantageously involve the use of a puncture locator device, as described in detail herein, to identify the ideal/target site for needle and/or introducer insertion through the heart wall 18 and for facilitating insertion of the puncture needle along a desirable puncture/access path through the tissue wall 18.

Once the target site for puncture of the heart wall 18 is verified/identified, a needle, such as an 18-gauge needle (e.g., trocar) or the like, may be inserted into the target site/ventricle. At block 304, the process 300 involves inserting a needle 40 at the identified puncture site. For example, the needle shaft 40 may be pressed/advanced through the heart wall 18 to access the ventricle 3. In some examples, the needle 40 may be a hypodermic needle having an internal axial lumen.

At block 306, the process 300 involves advancing a guidewire 42 through a lumen of the needle shaft 40, to thereby access the ventricle 3 through the needle 40. In some implementations, when the needle 40 penetrates the ventricle 3, a 0.035″ guidewire is advanced through the needle under imaging (e.g., TEE) guidance.

At block 308, the process 300 involves proximally withdrawing the needle 40 to thereby remove the needle 40 from the heart wall 18 and from around the guide wire 42. The guidewire 42 is thereby left in place in the heart wall 18 and partially within the ventricle 3. It may be advantageous to relatively slowly remove the needle 40 to prevent injury/damage to the patient anatomy.

At block 310, the process 300 involves placing an introducer 200 over the guidewire 42. For example, the introducer 200 may be passed over the guidewire 42 and advanced into the left ventricle 3. As shown in image 411, the introducer 200 may be inserted with a dilator 228 and passed over the guidewire 42 and advanced into the left ventricle 3. The surgeon/practitioner can use one or more sutures to make a series of stiches in one or more concentric circles in the myocardium at the desired location to create a “purse-string” closure. The Seldinger technique, as embodied at least in part in the various steps of the process 300 described above, can be used to access the left ventricle in the area surrounded by the purse-string suture by puncturing the myocardium with the needle 40 with the guidewire 42 implemented in the lumen of the needle/trocar 18 and advancing the introducer 200 over the guidewire. Once the introducer 200 is properly placed, the purse-string suture can be tightened to reduce bleeding around the lumen of the introducer.

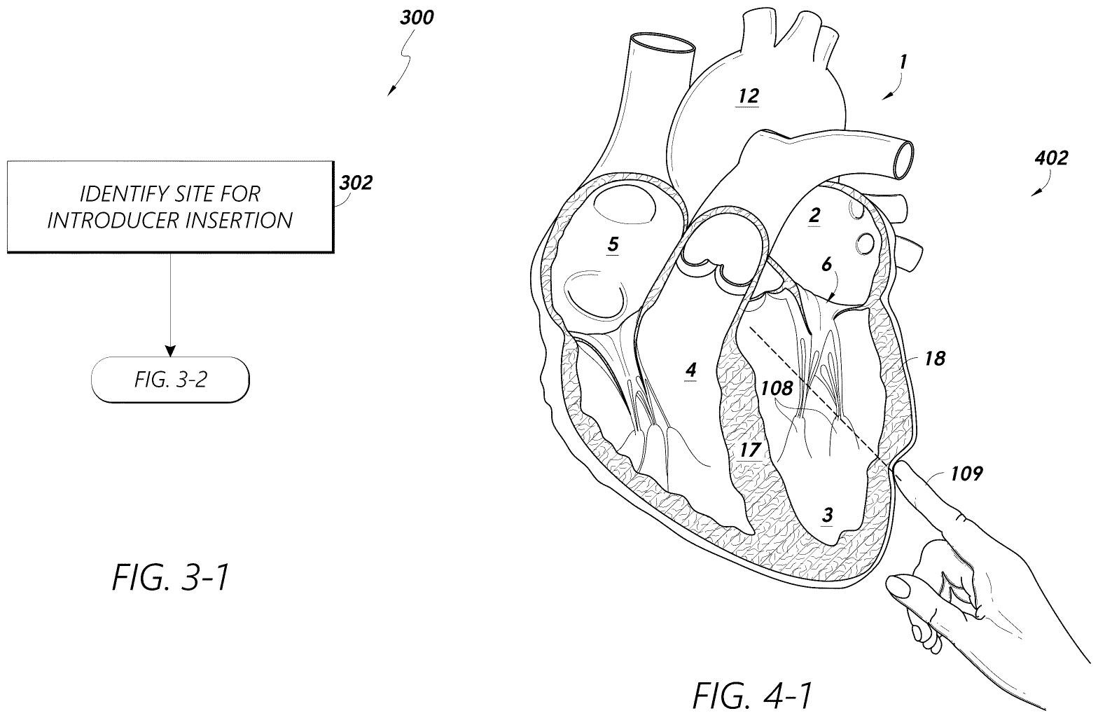

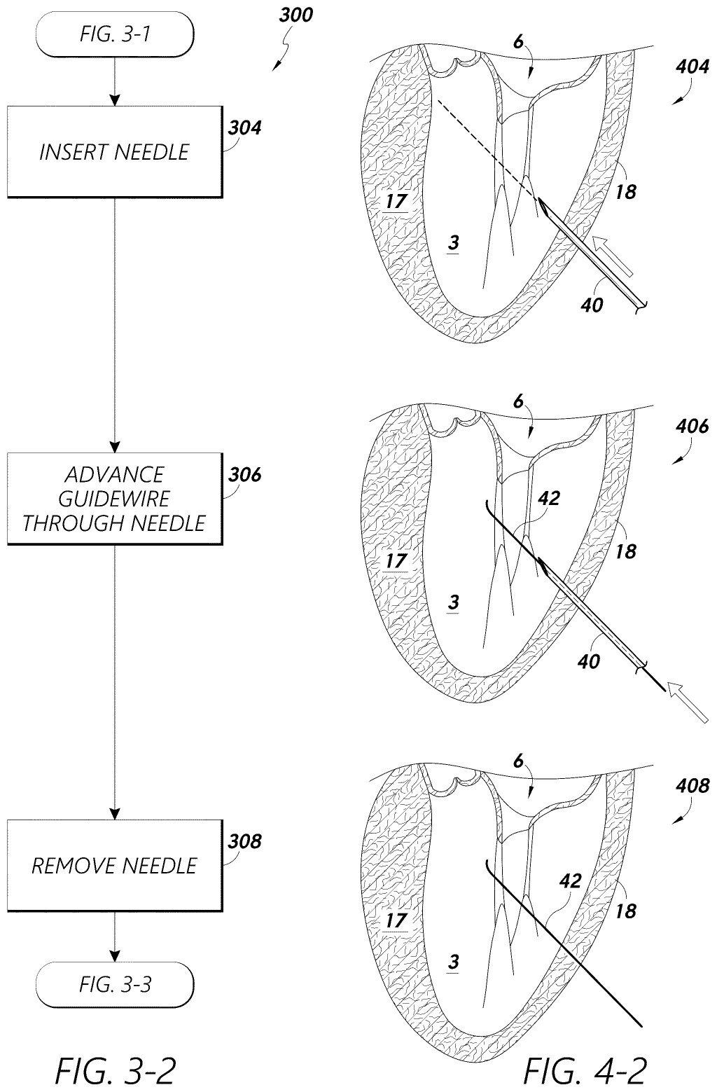

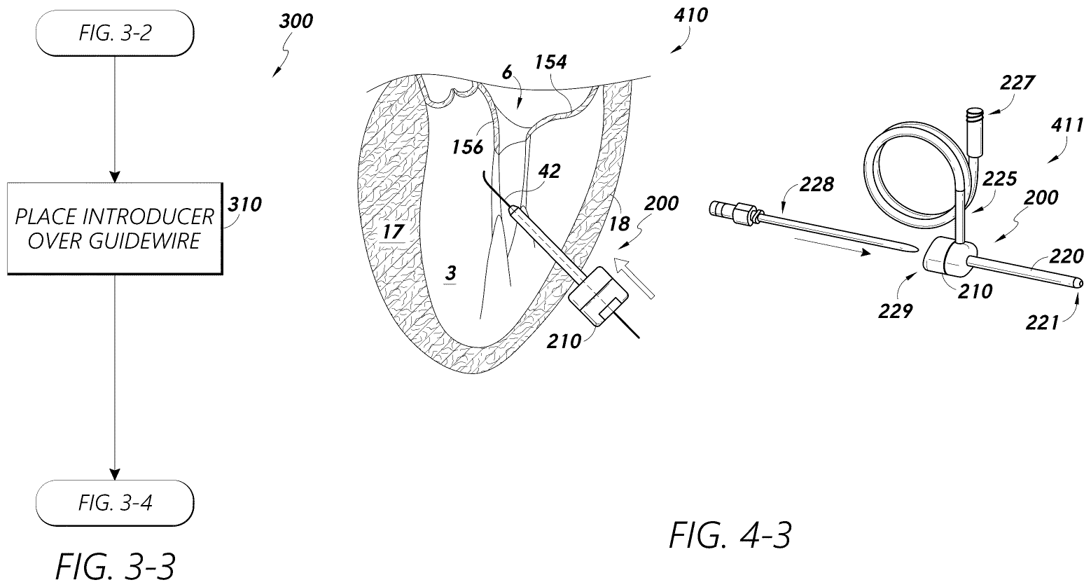

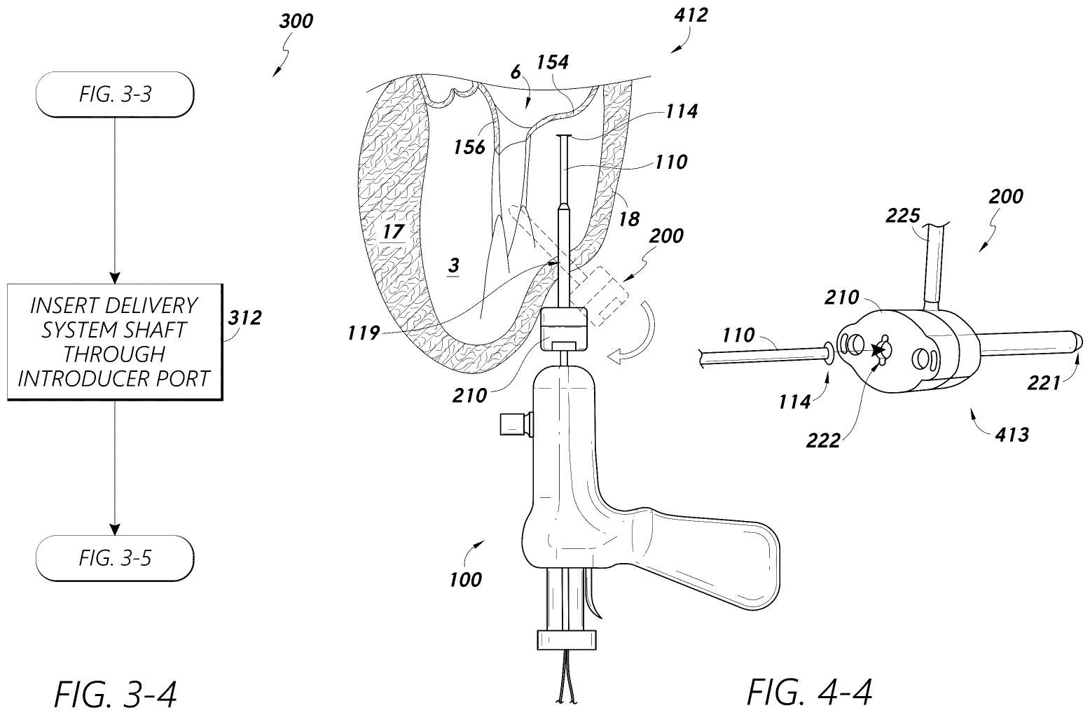

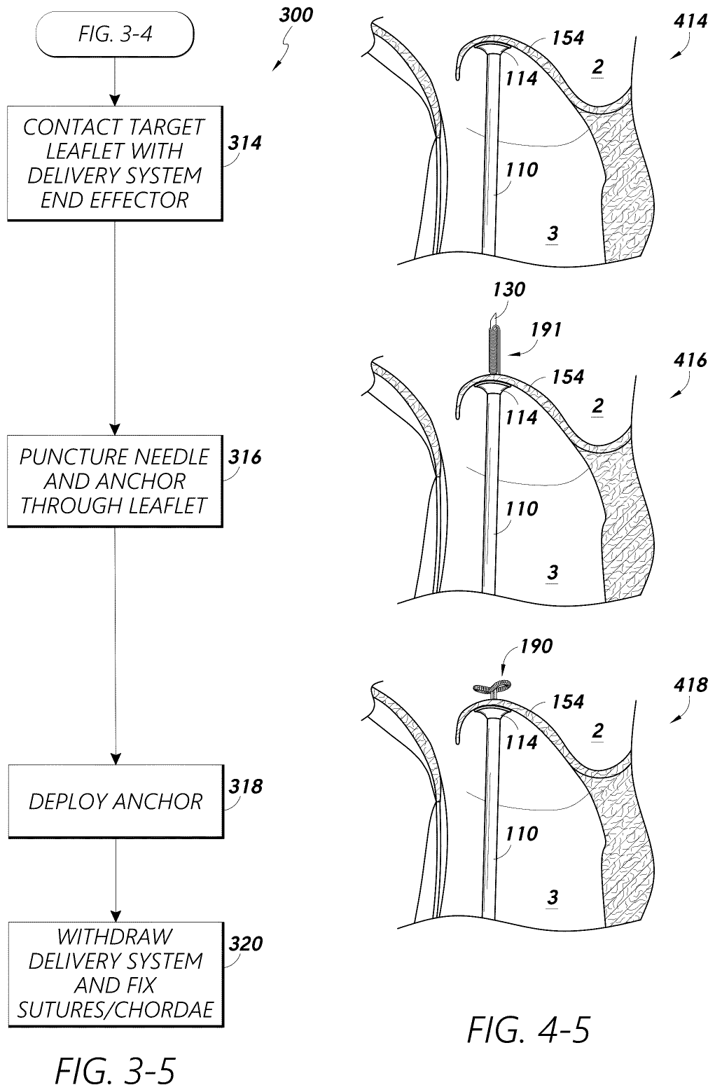

With respect to images 410-413 of FIGS. 4-3 and 4-4, respectively, the introducer/port device 200 may contain one or more fluid-retention valves to prevent blood loss and/or air entry into the ventricle 3. Image 411 shows a perspective view of a dilator 228 and introducer 200 that may be used in the process 300 in accordance with one or more examples. Image 413 shows the introducer device 200 and a tissue anchor delivery device shaft 110 in accordance with one or more examples.

The hemostatic introducer 200 may be inserted into the target ventricle at a tip 221 associated with a lumen 220 of the introducer 200. The lumen 220 of the introducer 200 may be used to guide the shaft 110 of the tissue anchor delivery device/system 100. The body or hub 210 of the introducer 200 may be used to secure the introducer 200 to the pericardium/epicardium of the heart for stable entry of the shaft 110 of the tissue anchor delivery device 100 and/or to control the amount of bleed-back during the process 300. In some instances, a female Luer may be used to de-air the introducer 200 through a port 225 prior to use and/or to connect a fluid flush, such as a heparin flush, during the process 300. The dilator 228 may be used to guide the introducer 200 into the target ventricle 3.

The introducer lumen 220 provides a conduit into the target surgical area/chamber 3. In some instances, the introducer 200 comprises one or more hemostasis valves associated with a channel/lumen port 222. Such hemostasis valve(s) may comprise silicone or other flexible material configured to keep blood from flowing out of the channel/lumen port 222. The port 222 may serve as a tissue anchor delivery device lumen insertion port, wherein an inserted delivery device shaft 110 may pass through the lumen 220 of the introducer 200 and out the distal end 221 thereof for access to the target chamber. The port 222 may further be dimensioned to accommodate insertion of the dilator device 228 used to guide the introducer into the target chamber (e.g., left ventricle, off-apex). The distal end 221 of the introducer 200 may have a tapered shape to seal against the delivery system lumen and to reduce trauma from insertion thereof.

Once the introducer 200 is positioned, eyelets (see image 413 of FIG. 4-4) may be sutured to the heart wall 18 and secured to a purse-string tourniquet. At block 312, the process involves inserting a delivery system shaft 110 through a port 222 and lumen 220 of the introducer to access the ventricle 3. In some examples, a sheath may be inserted through the introducer 200, through which the delivery system shaft 110 is advanced. In some implementations, an endoscope may first be advanced through the introducer 200 to visualize the ventricle 3, the valve 6, and/or the sub-valvular apparatus. By use of an appropriate endoscope, a careful analysis of the malfunctioning valve 6 may be performed. Each segment of each leaflet may be carefully assessed to determine its pliability, integrity, and motion. Based on this assessment, the practitioner can determine whether the valve can indeed be repaired or must be replaced. The motion of the leaflets 154, 156 can be classified as slightly dysfunctional, prolapsed, or restricted and based on this classification, the necessary steps of the repair can be determined.

The shaft 110 may present a relatively low-profile delivery device, which may be dimensioned to fit within the lumen 220 of the introducer 200. For example, the shaft 110 may be a 3 mm (9 Fr) shaft. Furthermore, the tip (e.g., end effector) 114 may advantageously be flexible to allow for insertion into the lumen 220 even where the lumen 220 has a smaller diameter than the extended diameter of the tip 114. The delivery device 100 may be similar to any of the devices/systems described in the '761 PCT Application and/or the '170 PCT Application. The advancement of the device shaft 110 may be performed in conjunction with echo imaging and/or direct visualization (e.g., direct transblood visualization). For example, the delivery device 100 may be advanced in conjunction with transesophageal echocardiogram (TEE) guidance or intracardiac echo (ICE) to facilitate and direct the movement and proper positioning of the device for contacting the appropriate apical region of the heart. Typical procedures for use of echo guidance are set forth in Suematsu.

As shown in image 412 of FIG. 4-4, when the introducer is not projected in a straight line towards the target leave that 154, it may be necessary to torque the introducer 200 to align with a more direct path to the target leaflet 154 from the puncture location 119 in the heart wall 18. Such torquing of the introducer 200 can cause at least temporary deformation of the heart wall 18, which may result in undue stresses on the wall 18 during the procedure and/or result in an implanted suture that includes unwanted/problematic stress point(s) in the wall 18 when implanted as shown and described in greater detail below in connection with FIG. 5.

At block 314, the process 300 involves contacting a target leaflet 154 with the end effector 114 of the delivery system 100. Image 414 of FIG. 4-5 shows the shaft 110 of the tissue anchor delivery device 100 positioned on the target valve leaflet 154 (e.g., mitral valve leaflet). For example, the target site of the valve leaflet 154 may be slowly approached from the ventricle side thereof by advancing the distal end of the shaft 110 along or near to the posterior wall of the ventricle 3 (e.g., left ventricle), without contacting the ventricle wall.

Once the tip 114 is positioned in the desired position, the distal end of the shaft 110 and the tip 114 may be used to drape, or “tent,” the leaflet 154 to better secure the tip 114 in the desired position, as shown in image 414. Draping/tenting may advantageously facilitate contact of the tip 114 with the leaflet 154 throughout one or more cardiac cycles, to thereby provide more secure or proper deployment of leaflet anchor(s). The target location may advantageously be located relatively close to the free edge of the target leaflet 154 to minimize the likelihood of undesirable intra-atrial wall deployment of the anchor. Navigation of the tip 114 to the desired location on the underside of the target valve leaflet 154 may be assisted using echo imaging, as described in detail herein. Echo imaging may be relied upon to confirm correct positioning of the tip 114 prior to anchor/knot deployment.

At block 316, the process 300 involves puncturing a needle 130 having a tissue anchor associated therewith through the leaflet 154. For example, with the shaft 110 positioned against the target leaflet 154, the plunger 140 of the tissue anchor delivery device 100 can be actuated to move the needle 130 and a pusher disposed within the shaft 110, such that the coiled sutureform portion 191 of the suture anchor slides off the needle 130. As the plunger 140 is actuated, a distal piercing portion of the needle 130 punctures the leaflet 154 and forms an opening in the leaflet. Image 416 of FIG. 4-5 shows a close-up view of the distal portion of the delivery device shaft 110, showing the needle 130 and tissue anchor sutureform 191 projected therefrom through the target leaflet 154 in accordance with one or more examples. In some instances, the needle 130 is projected a distance of between about 0.2-0.3 inches (e.g., between about 5-8 mm), or less, distally beyond the distal end of the shaft 130 (e.g., beyond the tip 114). In some instances, the needle 130 is projected a distance of between about 0.15-0.4 inches (e.g., between about 3-10 mm). In some instances, the needle 130 is projected a distance of about 1 inch (e.g., about 2.5 cm), or greater. In some instances, the needle 130 extends until the distal tip of the needle and the entire coiled sutureform 191 extend through the leaflet 154. While the needle 130 and sutureform 191 are projected into the atrial side of the leaflet 154, the shaft 110 and tip 114 advantageously remain entirely on the ventricular side of the leaflet 154.

As the pusher (not shown) within the tissue anchor delivery device shaft 110 is moved distally, a distal end of the pusher advantageously moves or pushes the distal coiled sutureform 191 (e.g., pre-deployment coiled portion of the suture anchor) over the distal end of the needle 130 and further within the atrium 2 of the heart on a distal side of the leaflet 154, such that the sutureform extends distally beyond a distal end of the needle 130. For example, in some instances, at least half a length of the sutureform 191 extends beyond the distal end of the needle 130. In some instances, at least three quarters of the length of the sutureform 191 extends beyond the distal end of the needle 130. In some instances, the entire coiled sutureform 191 extends beyond the distal end of the needle 130.

At block 318, the process 300 involves deploying a tissue anchor 190 on the atrial side of the leaflet 154. For example, after the sutureform 191 has been pushed off the needle 130, pulling one or more of the suture tail(s) 195 (e.g., suture strands extending from the coiled portion of the suture) associated with the tissue anchor 190 proximally can cause the sutureform 191 to form a bulky knot anchor 190, as shown in image 418, which provides a close-up view of the formed suture anchor 190 on the atrial side of the leaflet 154. For example, the bulky knot suture anchor 190 may be formed by approximating opposite ends of the coils of the sutureform 191 towards each other to form one or more loops. After the sutureform 191 has been formed into the bulky knot 190, the delivery device 100 can be withdrawn proximally, leaving the tissue anchor 190 disposed on the distal atrial side of the leaflet 154. In some instances, two suture tails 195 may extend from the proximal/ventricle side of the leaflet 154 and out of the heart. For example, the delivery device shaft 110 can be slid/withdrawn over the suture tail(s) 195.

At block 320, the process 300 involves withdrawing the delivery system and fixing/tying the artificial chordae/sutures associated with the tissue anchor 190 to the heart wall 18, to thereby tether the leaflet 154 to the heart wall 18 for the purpose of reducing or preventing leaflet prolapse.

In some implementations, echo imaging, such as involving TEE (two-dimensional (2D) and/or three-dimensional (3D)), transthoracic echocardiography (TTE), ICE, and/or cardio-optic direct visualization (e.g., via infrared vision from the tip of a 7.5 F catheter), or other imaging modality, may be performed to assess the heart, heart valves, and/or tissue anchor delivery device component(s) in connection with any of the steps of the process 300. For example, echo imaging can be used to guide positioning of tissue anchor(s) (e.g., suture knots) onto a target valve leaflet. Although the procedures described herein are with reference to repairing a cardiac mitral valve or tricuspid valve by the implantation of one or more leaflet anchors and associated cord(s), the methods presented are readily adaptable for various types of tissue, leaflet, and annular repair procedures. The methods described herein, for example, can be performed to selectively approximate two or more portions of tissue to limit a gap between the portions. That is, in general, the methods herein are described with reference to a mitral valve but should not be understood to be limited to procedures involving the mitral valve.

FIG. 5 shows a cutaway view of a deployed leaflet anchor 190 tethered to a ventricular wall 18 of a heart 1 in accordance with one or more examples. The suture tails 195 coupled to the anchor 190 may be secured at the desired tension using a pledget 71 or other suture-fixing/locking device or mechanism on the outside of the heart through which the suture tails 195 may run. Furthermore, one or more knots or other suture fixation mechanisms or devices may be implemented to hold the sutures at the desired tension and to the pledget 71. With the suture tail(s) 195 fixed to the ventricle wall 11, a ventricular portion 195a of the suture tail(s) 195 may advantageously function as replacement leaflet cords (e.g., chordae tendineae) that are configured to tether the target leaflet 54 in a desired manner. In certain implementations, testing of location and/or tension of the anchor 190 and/or suture tail(s) 195 may be performed by gently tensioning the suture tails until leaflet motion is felt and/or observed. Echo imaging technology may be used to view and verify the anchor placement and resulting leaflet function.

The present disclosure relates to the use of puncture locator devices for the purpose of locating and executing tissue punctures that are in-line, or relatively close to in-line, with a direct path between the puncture location in/on the epicardium/outer wall of the heart and the target tissue anchor deployment position (e.g., target mitral valve leaflet). Such puncture locating functionality can prevent undue strain, stress, tearing, or other damage or injury with respect to the tissue wall 18 and/or the target tissue/leaflet 154. For example, when the puncture path through the heart wall 18 is oriented/angled along a puncture path that deviates substantially from the direct path between the puncture location and the target tissue anchor deployment position, as in FIG. 5, a strong risk can exist of stress points in the myocardium as the suture makes a turn toward the epicardium in the heart wall 18, as shown in FIG. 5. Furthermore, such stress points can result in damage to the sutures 195, thereby introducing risk of breakage and/or other related consequences.

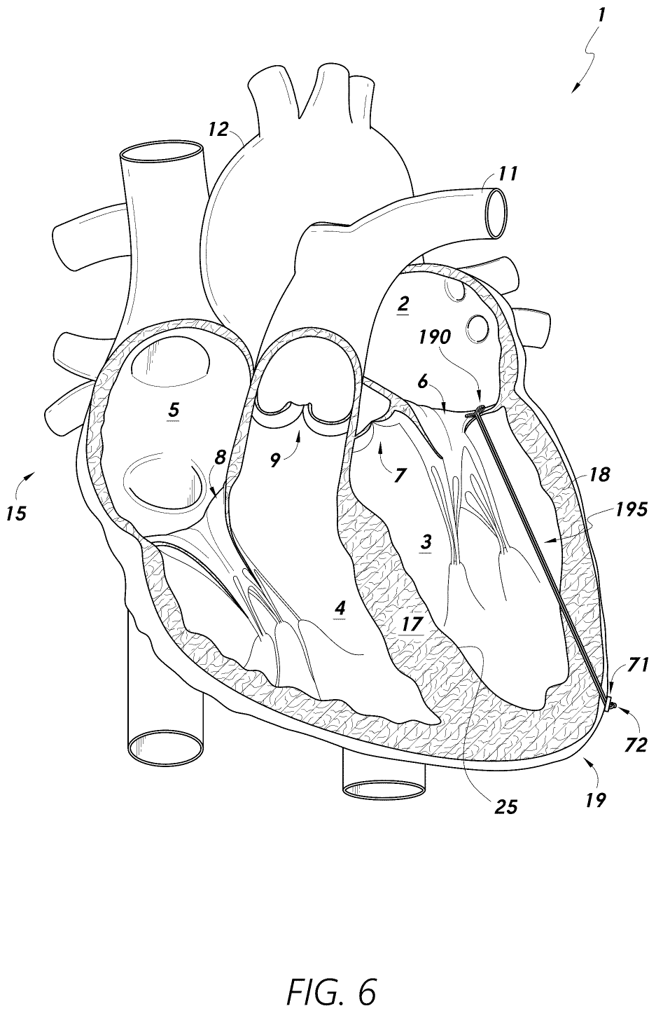

FIG. 6 shows a cutaway view of a deployed leaflet anchor 190 in a heart in accordance with one or more examples. With the use of a puncture locator device as described in detail herein, the suture path can be improved to assume a more straight line from the target valve leaflet to the epicardial surface, thereby potentially eliminating one or more stress points that could result in tissue damage or injury or undue stress on the suture(s) 195. The image of FIG. 6 corresponds to a puncture path executed using a puncture locator device and/or associated procedure according to examples of the present disclosure, and may produce a safer and or more effective leaflet tethering compared to leaflet anchors deployed without the use of such an aid.

The steps and processes outlined above for placing a suture-knot-type tissue anchor may be repeated as necessary until the desired number of anchors have been implanted on the target valve leaflet(s). For example, a plurality of leaflet anchors can be deployed in each of the mitral valve leaflets. In some implementations, sutures/cords coupled to separate leaflets may be secured together in the heart by tying them together with knots or by another suitable attachment device, creating an edge-to-edge repair to decrease the septal-lateral distance of the mitral valve orifice.

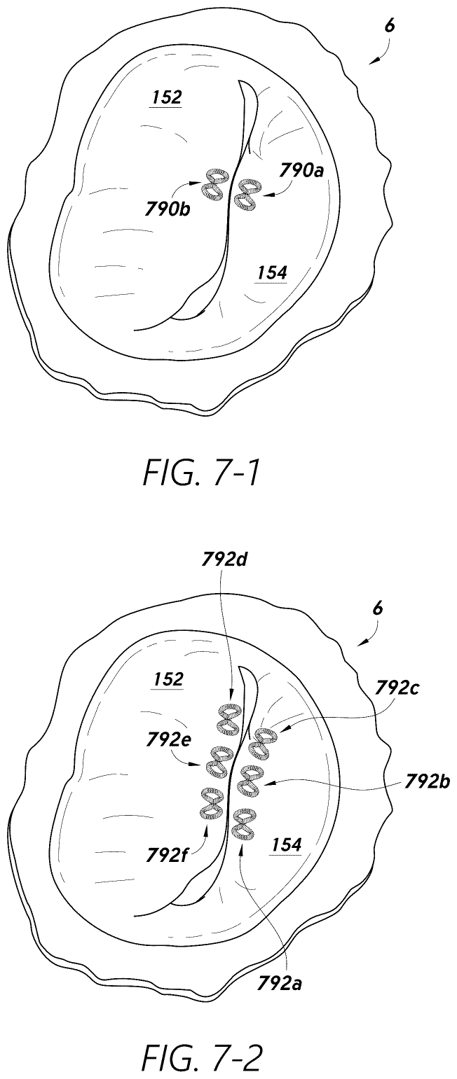

FIG. 7-1 illustrates an atrial/distal side of a valve (e.g., viewing the mitral valve from the left atrium) having a plurality of tissue anchors deployed therein according to one or more instances disclosed herein. FIG. 7-1 shows two tissue anchors 790 with a bulky knot form delivered to the atrial/distal side of the valve 6. As shown in FIG. 7-1, a first tissue anchor 790a may be delivered to a first leaflet 154 and/or a second tissue anchor 790b may be delivered to a second leaflet 152. However, any number of tissue anchors may be delivered to either leaflet. For example, the first tissue anchor 790a and second anchoring element 790b may alternatively both be delivered to the first leaflet 154 or the second leaflet 152.

FIG. 7-2 illustrates an overhead view of an upper/distal side of a valve having six anchoring elements with a bulky knot form delivered to the upper/distal side of the valve according to one or more instances disclosed herein.

FIG. 7-2 illustrates a view of an atrial/distal side of a valve having six tissue anchors 792 deployed therein according to one or more examples. In some instances, the six anchoring elements 792 may be delivered simultaneously via a single delivery device (e.g., a needle) and/or may be deployed sequentially. As shown in FIG. 7-2, a first tissue anchor 792a, second tissue anchor 792b, and/or third tissue anchor 792c may be delivered to a first leaflet 154 and/or a fourth tissue anchor 792d, fifth tissue anchor 792e, and/or sixth tissue anchor 792f may be delivered to a second leaflet 152. However, any number of tissue anchors 792 may be delivered to either leaflet.

The appropriate number of leaflet anchors may advantageously be determined to produce the desired coaptation of the target valve leaflets 154, 152. Some or all deployed leaflet anchors may advantageously be below the surface of coaptation. With respect to posterior mitral valve leaflet repair, the anterior leaflet 152 may advantageously touch the posterior leaflet 154 basal to the leaflet anchor(s). In some implementations, tension adjustment in the suture tail(s)/cord(s) associated with multiple leaflet anchors may be performed simultaneously. For example, a pledget may be drawn against the epicardial surface, and all the suture tails/cords may be inserted through a tourniquet so that all cords can be tension to the desired effective coaptation together.

FIGS. 8A-8G provide views of a puncture/access locator device 50 in accordance with one or more examples. The puncture locator device 50 (as well as the puncture locator device 60 shown in FIGS. 9A-9E) can provide a device with features that function collectively as a localization instrument configured to facilitate identification of a specific target puncture site on, for example, the left ventricle wall for needle, and ultimately introducer, insertion. Puncture locator devices in accordance with aspects of the present disclosure can allow for improved methods for quickly and safely inserting, positioning, and advancing access needles and introducers for tissue anchor deployment processes as disclosed herein.

FIG. 8A shows a top, back, and side perspective view of the puncture locator device 50, wherein the puncture locator device 50 is shown with a needle 40 disposed therein. For example, the needle 40 may include a shaft 48 having a distal pointed tip 49, as well as a proximal handle form or structure 46 on an opposite end of the needle 40 from the pointed tip 49. The puncture locator device 50 includes a manual manipulation means 52, such as a handle member, which may be proximally-projecting from a stabilizer means (e.g., structure/base) 54 of the device 50. For example, with respect to the puncture locator device 50, the proximal direction may generally be aligned with the axis A₁ of the handle 52 and projecting in a direction towards the user when the user is manually holding or manipulating the puncture locator device 50. For example, the side view shown in FIG. 8C illustrates general directionality of the proximal and distal directions/orientations relative to the puncture locator device 50 with respect to the description thereof herein.

FIG. 8B shows a bottom, front, and side perspective view of the puncture locator device 50. FIG. 8C shows a side view of the puncture locator device 50, whereas FIGS. 8D and 8E show back and front views, respectively, of the puncture locator device 50. FIGS. 8F and AG show top and bottom views, respectively, of the puncture locator device 50.

The puncture locator device 50 includes a tissue depressor means 55 (e.g., form or member), which may protrude from an underside 57 of the stabilizer structure 54. For example, the tissue depressor form 55 may comprise a dome and/or boss form projecting from a plane P₁ associated with the stabilizer structure 54. Therefore, in some examples, the underside 57 of the stabilizer structure 54 may have the tissue depressor form 55 project distally therefrom, whereas the handle 52 may project proximally from the top side 59 of the stabilizer structure 54. Although the handle member 52 is shown as projecting from a central area of the stabilizer structure 54, in some examples, the handle member 52 may project from a back or front portion of the stabilizer structure 54 (e.g., of the top surface 59). The puncture locator device 50 can guide a needle through a needle channel 56 formed in and/or otherwise associated with the puncture locator device 50 into tissue at an ideal/desired puncture site. The tissue depressor form 55 and the stabilizer structure (e.g., under/distal/bottom side thereof can collectively be referred to as, or considered, a tissue-contact structure.

The bottom/ventral surface 57 may be integrated with the tissue depressor form 55. The tissue depressor form/feature 55 can have any suitable or desirable shape or form. For example, the tissue depressor form 55 can comprise a relatively small bulbous, dome, and/or cone-shaped structure with rounded corners. In some examples, the tissue-depressor form 55 has a diameter D₂ that is approximately half the diameter of an average adult human index finger.

Although shown as having a substantially flat top surface 59, it should be understood that the stabilizer structure 54 may have any shape or form. The stabilizer structure 54 may serve to orient the puncture locator device 50, and/or the handle 52 and/or the tissue depressor form 55 thereof, in an orientation substantially normal or orthogonal to a tissue surface against which the distal portion of the puncture locator device 50 may be held when used to facilitate puncture location/access as described in detail herein. The underside/surface 57 of the stabilizer structure 54 can serve as a tissue contact surface, at least with respect to a perimeter thereof, when the tissue depressor form 55 is pressed against a target tissue wall. Therefore, the stabilizer structure 54 may generally have a diameter D₁ that is greater than either or both of the diameter D₂ of the tissue depressor form 55 or the diameter D₃ of the handle 52 in one or more dimensions. With respect to implementations of the tissue depressor form 55 as a sloped dome as in the embodiment of FIGS. 8A-8G, the diameter of the tissue depressor form 55 may be understood to be the diameter of the circle or ellipse associated with the inflection point of the curvature of the sloped sides of the dome around the tissue depressor form 55. That is, as can be seen in FIG. 8C, although the tissue-depressor form 55 expands out to near the perimeter of the stabilizer structure 54 at its base, the diameter of the tissue depressor form 55 may be understood to be a dimension smaller than the base of the depressor form, as shown.

The stabilizer structure 54 may have a diameter D₁ (e.g., front-to-back diameter D₁ₐ and/or side-to-side diameter D_{1b}) of any suitable or desirable length. For example, the diameter D₁ may be in the range of 2-3 cm, such as about 2.5 cm. Furthermore, the handle member 52 may have a diameter D₃ (e.g., at a proximal end 73 and/or base 75 thereof) of any suitable or desirable length. For example, the diameter D₃ may be in the range of 1.5-2.5 cm, such as about 1 cm. Furthermore, the tissue depressor form 55 may have a diameter D₂ of any suitable or desirable length. For example, the diameter D₂ may be in the range of 0.75-2 cm, such as about 1.5 cm. Furthermore, the handle member 52 may have a height H₁ of any suitable or desirable dimension, wherein the height H₁ may be understood to be a distance between the top 59 of the stabilizer structure 54 and the top/proximal end 73 of the handle member 52. For example, the height H₁ may be in the range of 2-8 cm, such as about 2.5 cm. Furthermore, the tissue depressor form 55 may have a height H₂ of any suitable or desirable dimension, wherein the height H₂ may be understood to be a distance between the bottom 57 of the stabilizer structure 54 and the apex 76 of the tissue depressor form 55. For example, the height H₂ may be in the range of 0.75-3 cm, such as about 1.5 cm.

The puncture locator device 50 includes a needle channel 56, which may run internally through one or more components of the puncture locator device 50, such as through portions of one or more of the handle member 52, the stabilizer structure 54, and the tissue depressor form 55. The needle channel 56 may be disposed in a block or portion of the device 50 positioned above the tissue depressor form 55, and may comprise a hole, aperture, and/or channel that may be reamed, drilled, milled, molded, or otherwise formed in the structure of the device 50. As referenced herein, the needle channel 56 may pass completely through from a backside to front side of the device 50 and may be designed as a track for aligning and directing a needle inserted therein.

The needle channel 56 includes a proximal opening 58, into which the tip 49 of the needle 40 can be inserted on a proximal and back side/portion of the device 50. The needle channel 56 further includes a distal opening 51 through which the tip 49 of the needle can exit the needle channel 56 on a distal and front side/portion of the device 50. The distal opening 51 of the needle channel 56 is advantageously associated with and/or disposed on a front side surface 79 of the tissue depressor form 55. The position of the distal opening 51 of the needle channel 56 on the front side surface 79 of the tissue depressor form 55 may allow for the needle channel to guide the needle 48 at an acute, non-zero angle θ₁ with respect to the plane P₁ of the stabilizer structure 54. For example, the plane P₁ of stabilizer structure 54 may be generally orthogonal or perpendicular to one or both of the axis A₁ of the handle member 52 and/or the axis A₂ of the tissue depressor form 55. Therefore, the needle channel 56 may advantageously be angled with respect to the axis A₁ of the handle member 52 and/or axis A₂ of the tissue depressor form 55, as shown. In some examples, the axes A₁ and A₂ are substantially the same and/or aligned. In some examples, the axes A₁ and A₂ are parallel, but offset/unaligned. The angle θ₁ may be selected to create a desired/optimum entry angle for needle puncture through the tissue wall (e.g., ventricle wall), such that the puncture path provides a relatively straight path for the introducer to subsequently enter the ventricle through the needle puncture path towards the target anatomy (e.g., mitral valve).

The needle channel 56, which is designed as a needle guide, can be circular in cross-section through at least a portion of the length thereof. However, it should be understood that the needle channel can assume a rectangular or square shape in one or more portions/lengths thereof. In some examples, the interior surfaces of the needle channel 56 may include longitudinally/axially oriented/running corrugations, ridges, channels, peaks, or other features configured to limit the circumferential contact surface of the needle shaft 48 with the needle channel 56 to thereby reduce frictional resistance on the needle shaft 48 when inserting/advancing the needle 40. For example, such shapes can serve to limit the surface contact between the needle shaft 48 and the needle channel 56 to certain longitudinal line(s) or band(s), thereby presenting a relatively lower frictional resistance between the needle shaft 48 and the needle channel 56 compared to some smooth cylindrical channel implementations, wherein the frictional force/resistance is proportional to the surface contact area between the channel 56 and the needle shaft 48. Longitudinal corrugations, as described above, may further facilitate an effective and secure hold of the needle shaft 48 in the oriented position within the needle channel 56. The needle channel 56 advantageously provides a predetermined angle of insertion for the needle 40, potentially removing variability in procedural implementation, thereby providing more consistent and/or effective results.

In some examples, the handle member 52 may have associated therewith a needle stopper and/or alignment feature 38, which may be configured to limit distal insertion of the needle 40 by contact between the stoppers/alignment feature 38 and the handle portion 46 of the needle 40. For example, the stoppers/alignment feature 38 may project radially from a circumferential portion of the handle member 52, such as from a perimeter of the proximal end portion 73 of the handle member 52. The stopper/alignment feature 38 may help control the advancement and desired depth of needle penetration by limiting the distal movement of the needle 40. In some examples, the needle channel 56 extends proximally from the handle 50 by some distance, wherein the proximal opening 58 of the needle channel 56 serves as a stopper limit for the needle 40. That is, the needle channel may comprise a projection or extension of the handle 52 or other structure of the device 50 (e.g., stabilizer 54) that serves to contain the needle channel 56 and extend the needle channel farther proximally than is shown in FIGS. 8A-8G to set the stopper position for the needle handle 46.

In the embodiment of FIGS. 8A-8G, the needle channel 56 is shown as passing through a distal portion of the handle member 52, as well as through an area in the center of the stabilizer structure 54 and through at least base and frontside portions of the tissue depressor form 55. The needle channel 56 may be dimensioned to allow for insertion therethrough of a hypodermic needle and may have a generally cylindrical inner diameter/surface. The needle channel 56 can advantageously have minimal clearance around the needle shaft 48, to thereby restrict the orientation of the needle shaft 48 when disposed in the needle channel 56.

The stabilizer structure 54 includes an orientation indicator feature 53, which may comprise a point/apex feature and/or other form or feature, wherein such orientation indicator 53 can serve to facilitate alignment of the puncture locator device 50 by the user. For example, the user may manually manipulate the handle 52, such as by turning and/or rotating the handle 52 and/or device 50 to thereby point or align the orientation indicator 53 towards/with target anatomy that is being targeted by the needle 40 for puncture. For example, with respect to target anatomy including a heart valve leaflet within a ventricle and/or atrium of the heart, the surgeon may point the orientation indicator 53 in the general direction of the target leaflet/valve to thereby orient the needle channel 56 in a direction that facilitates access to such target anatomy. The point/indicator of the orientation indicator feature may be aligned with and/or lie within a plane P₃ associated with the needle channel 56, as shown in FIG. 8E.

As best shown in FIGS. 8A, 8B, and 8G, in some examples, the stabilizer structure 54 has a shape indicative of an anatomical feature associated with the intended utility of the puncture locator device 50. For example, as shown, the stabilizer structure 54 may have a heart shape, indicating use for targeting anatomy of the heart and/or for placement on/against the heard. In such heart-shaped configurations, the apex 53 of the heart may serve as the orientation indicator feature described above. The heart shape may be represented by the top surface 59, bottom/ventral surface 57, and/or other surface or portion of the stabilizer structure 54. Although illustrated with a stabilizer structure 54, it should be understood that in some instances, no stabilizer structure 54 is included, such that the tissue depressor form 55 projects from a base of the handle member 52, without additional stabilizing structure.

In addition to the design benefit of the heart-shaped stabilizer 54, the heart shape may further provide certain utility with respect to stabilization functionality. For example, the mirrored cusps 78 at the back of the stabilizer associated with the heart shape can provide wing-type stabilization on either side of the device 50 on the backside thereof, such that the cusps 78 serve as stabilizing feet for tissue contact and stabilization to decrease or prevent lateral (e.g., side-to-side) tilting of the device 50. Although shown as a heart-shaped structure, it should be understood that the stabilizer structure 54 may have any shape or form, such as a relatively small, compact block design, such as a wedge-/platform-type form or the like.

FIGS. 9A-9E provide views of a puncture/access locator device 60 in accordance with one or more examples. The puncture locator 60 shown in FIGS. 9A-9E may be similar in certain respects to the puncture locator 50 shown in FIGS. 8A-8G. Therefore, any of the description of FIGS. 8A-8G may apply to the illustrated embodiment of FIGS. 9A-9E, and vice versa.

The puncture locator device 60 includes a handle member 62 coupled to and/or projecting from a stabilizer structure 64. In the embodiment shown in FIGS. 9A-9E, the handle member 62 projects generally from a back portion of the stabilizer structure 64. The handle 62 may include a base portion 71, which may provide stabilizing functionality for the handle 62 and/or structure for housing the needle channel 66. The base 71 of the handle member 62 may extend and/or project forward from the base of the handle 62, as shown, which may provide increased purchase for the handle member 62 with the stabilizer structure 64, thereby strengthening the coupling/integration between the handle member 62 and the stabilizer structure 64.

In some examples, the handle 62 may include an aperture feature 72, wherein the needle shaft 48 of the needle 40 may be inserted into the proximal opening 68 of the needle channel at least partially within or through the aperture feature 72 of the handle 62. For example, the aperture feature 72 may be open to expose the proximal opening 68 of the needle channel 66, and may thereby provide a window in the handle 62 for needle passage. In some examples, the inside walls of the aperture feature 72 have/present a corrugated surface, which may provide enhanced structural strength for the handle 62.

The needle channel 66 may extend through the base 71 of the handle member 62, and through at least a portion of the stabilizer structure 64 and tissue depressor form 65 projecting from a bottom/ventral side 67 of the stabilizer structure 64. The needle channel outlet 61 is positioned on a front side 75 of the tissue depressor form 65.

In the embodiment of FIGS. 9A-9E, the handle 62 may be relatively narrow front-to-back compared to the handle 52 shown in FIGS. 8A-8G, as shown. Such narrow dimension may allow for manipulation of the handle 62 within a relatively tight opening in the chest wall of the patient, while still allowing the surgeon to impose sufficient/desired leverage on the device 62 to orient the device as desired. Therefore, the implementation of the handle 62 in which the handle is wider than it is thick, as shown in FIGS. 9A-9E can be beneficial for manipulation of the device 60. For example, the front-to-back thickness D₄ of the handle 62 may be in the range of about 0.5-1.5 cm, such as about 1 cm.

Furthermore, as with any of the examples disclosed herein, the height of the handle 62 may advantageously be long enough to span the anatomy between the exterior wall of the heart and the skin of the patient, such that the surgeon can manually manipulate the device from outside of the patient, and without substantially obstructing the field of view available to the surgeon. For example, the handle 62 may have a height H₃ in the range of 3-6 cm, such as about 4 cm. The tissue depressor form 65 may be positioned substantially in the center of the stabilizer structure 64, or may be positioned in a biased position towards the front of the stabilizer structure 64 in some examples.

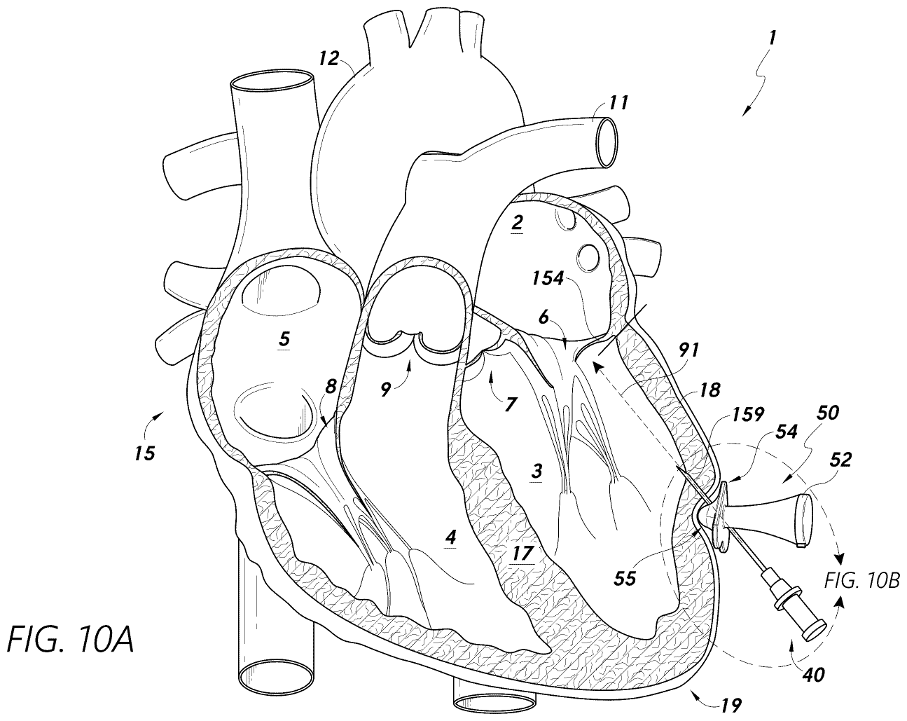

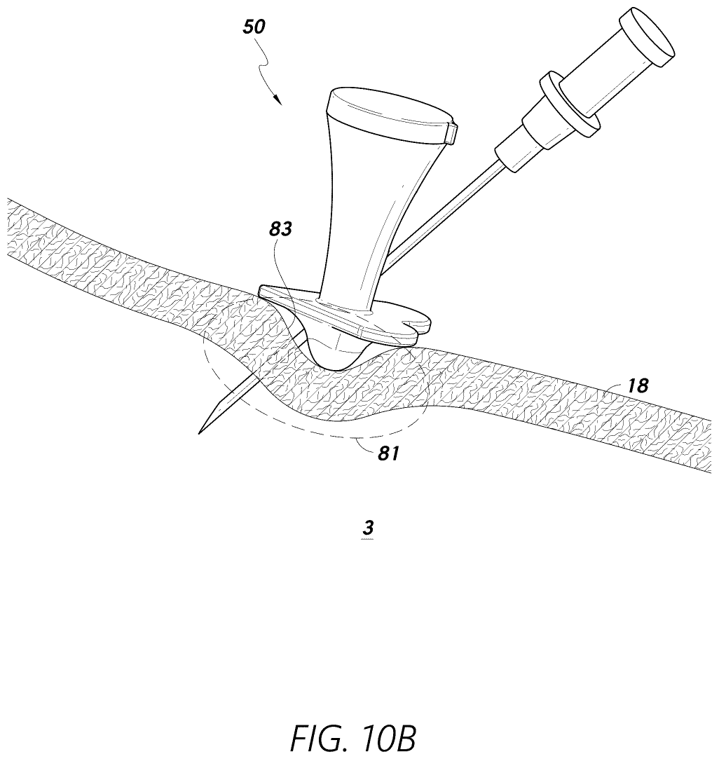

FIGS. 10A and 10B show a puncture/access locator device 50 pressed against a heart wall in accordance with one or more examples. FIG. 10A shows the puncture locator device 50 placed against a heart wall 18 such as an outer epicardium of a ventricle of the heart 1, such as the left ventricle 3. FIG. 10B shows a close-up view of the puncture locator device 50 against the heart wall 18. The puncture locator device 50 may be used to puncture the heart wall 18 along a puncture line 91 projecting towards target anatomy within the heart 1, such as a leaflet 154 of a heart valve 6 (e.g., posterior leaflet of mitral valve).

With the distal portion of the puncture locator device 50 pressed against the heart wall 18, the tissue depressor form 55 may depress and/or indent the outer wall 18 to produce a concavity in the heart wall 18, as shown. With the heart wall depressed, as shown, the needle channel 56 of the puncture locator device 50 may project incident with an inclined wall 83 caused/presented as a result of the depression of the wall 18, wherein the inclined wall 83 may present a puncture location 159 and/or path that is ideal or otherwise desirable for accessing the target tissue/leaflet. For example, as shown in FIG. 10A, the needle channel 56 may provide a puncture path through the tissue wall 18 that projects along a substantially straight, or relatively near-straight, line 91 to the target tissue/leaflet (e.g., posterior mitral valve leaflet) from the puncture location 159.

The puncture locator device 50 can serve to direct the needle 40 in a relatively straight vector towards the target anatomy (e.g., mitral valve) from the ventricular epicardium. Providing a straight vector for suture implants between target leaflet 154 and the puncture access 159 can help reduce stress and/or stress points between the ventricle wall 18 and the tissue anchor implant implanted in the target leaflet 154. Deflecting the heart wall 18 through depression thereof as shown in FIGS. 10A and 10B can allow for an angled introduction of the needle 40 into the ventricle in the direction of the target anatomy.

FIG. 11 shows a puncture/access locator device 50 placed against a heart wall 18 with an orientation indicator 53 of the device oriented in a direction of a target anatomy in accordance with one or more examples. The puncture locator device 50 includes a proximal handle 52, a stabilizer structure 54, and an orientation indicator/pointer 53, which may indicate an alignment of a needle channel associated with the puncture locator device 50.

In use, with respect to a procedure for targeting a target heart valve leaflet, the surgeon/practitioner may place the distal portion of the puncture locator device 50 against the exterior wall 18 of the heart 1 and manually manipulate the device 50 to point the orientation indicator/pointer 53 and the estimated direction of the target anatomy (e.g., mitral valve leaflet). For example, the orientation indicator/pointer 53 may indicate a line/vector 92, wherein the surgeon/practitioner may manipulate the handle 52 to bring such line/vector 92 into alignment with the target anatomy. Imaging guidance may be implemented to assist such alignment. In some implementations, the device 50 may be placed lateral to (e.g., to the right of) the lateral to the left anterior descending artery (LAD) 111, which may serve as a prominent visual line of demarcation and reference for placement of the puncture locator device 50.

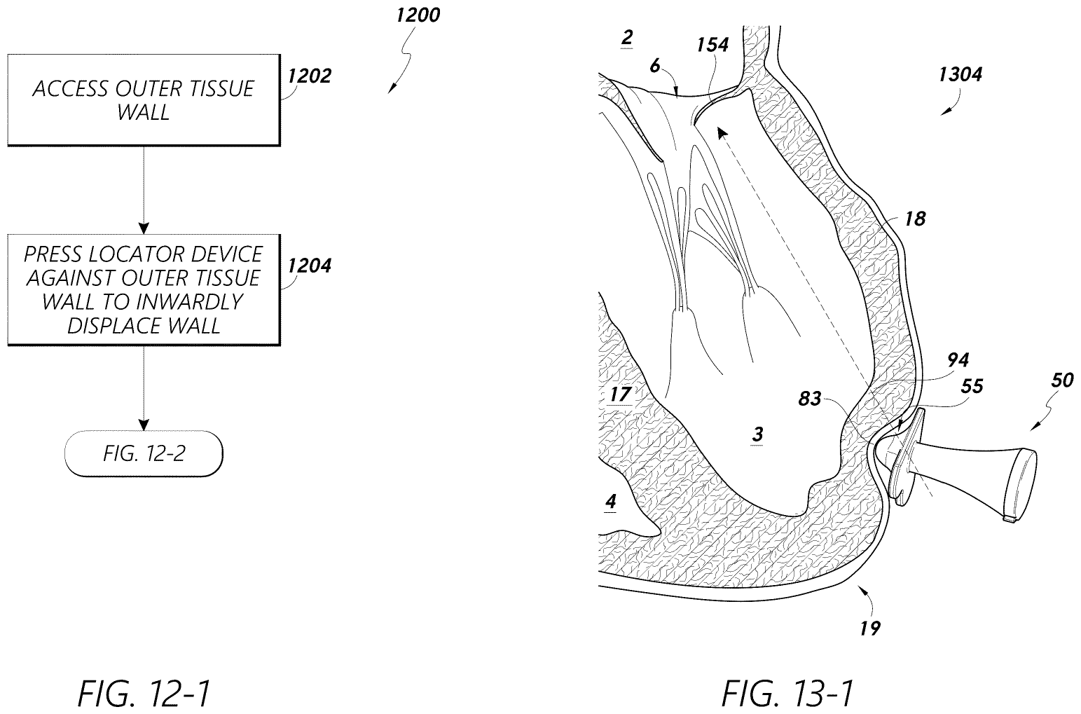

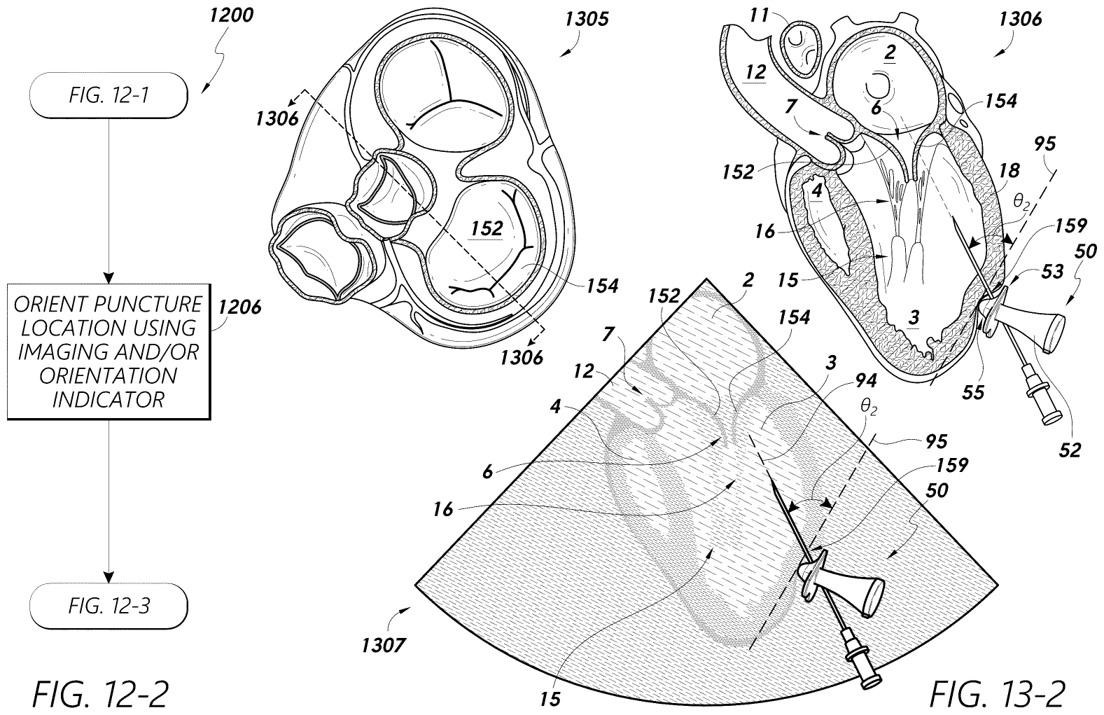

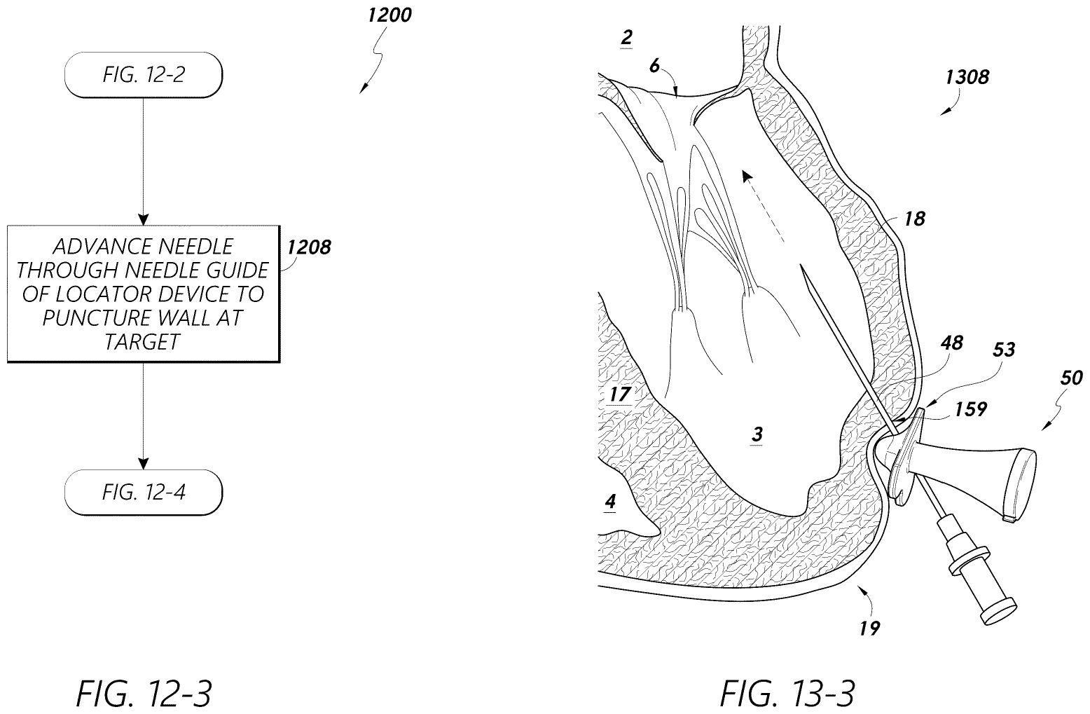

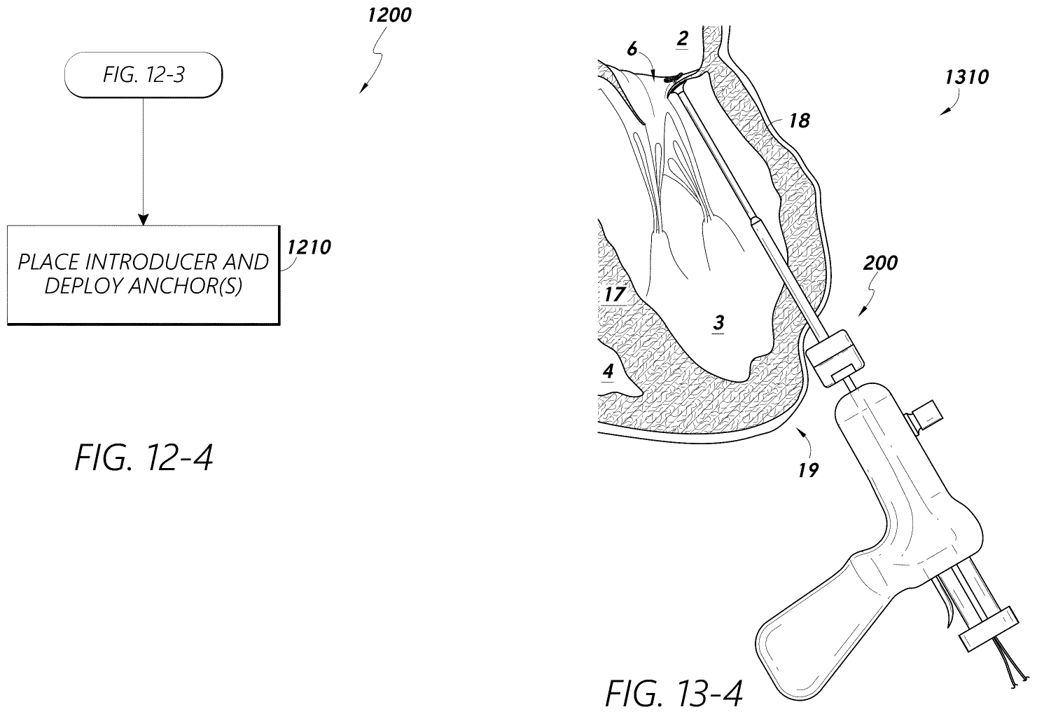

FIGS. 12-1, 12-2, 12-3, and 12-4 provide a flow diagram illustrating a process 1200 for puncturing a tissue wall using an access locator device in accordance with one or more examples. FIGS. 13-1, 13-2, 13-3, and 13-4 provide images of cardiac anatomy and certain devices/systems corresponding to operations of the process 1200 of FIGS. 12-1, 12-2, 12-3, and 12-4 in accordance with one or more examples. At block 1202, the process 1200 involves accessing an outer tissue wall, such as a pericardium of a patient's heart. Such access may be made through the chest wall in some cases, or through other minimally-invasive means.

At block 1204, the process 1200 involves pressing a puncture locator device 50 against the heart wall 18 to inwardly displace the wall with a tissue depressor form 55 projecting from a distal end portion of the puncture locator device 50. Such displacement may advantageously present an angled wall portion 83 providing a puncture path 94 projecting to a target valve leaflet 154 (e.g., posterior mitral valve leaflet). Although shown without a needle disposed therein in image 1304, in some implementations, the device 50 may be preloaded with a needle when initially pressed against the heart wall 18. For example, the needle may be partially inserted in the needle channel of the device 50 in a position in which the point of the needle does not protrude from the distal opening of the needle channel. For example, the needle may be disposed in a position wherein the tip thereof does not protrude from the distal opening of the needle channel, wherein such disposition can decrease the risk of accidental needle prick injury to the user and/or patient anatomy.

During operation, the device 50 can advantageously be handled and operated with one hand, such as by using a thumb and index finger with the handle 52 as the device 50 contacts the heart wall 18. To locate the access site, the surgeon/practitioner may use one or more digits to apply pressure on the handle 52 and/or top surface thereof, thereby pushing the tissue depression feature 55, which may have a dome-shaped structure/form, downward to displace the ventricular wall 18 inward. Such action may simulate a finger poke on the tissue wall, as described above.

At block 1206, the process 1200 involves orienting the puncture locator 50 using certain imaging technology and/or an orientation indicator 53 associated with the puncture locator device 50. For example, a depression of the heart wall 18 by the tissue depressor form 55 can be visible in an echo image 307 (e.g., long-axis), which may allow for the surgeon/practitioner to find the proper position and orientation for needle puncture. Furthermore, the handle component 52 may make it relatively easy for the surgeon/practitioner to maintain depression of the heart wall 18 while examining the echo imaging, thereby reducing instances of the surgeon/practitioner removing the depression and subsequently having to reposition/find the desired device/puncture position. In some examples, one or more components of the puncture locator device 50, such as the tissue depressor form 55, may have certain echogenic material or coding associated therewith for the purpose of allowing for visual identification thereof under echo imaging.

The handle component 52 may provide ease of handling of the device 50, such that the device 50 provides a relatively compact, and easily operated/manipulated device, wherein manipulation thereof may be implemented through relatively simple thumb or other digital pressing of the handle 52. Furthermore, the surgeon/practitioner may be able to relatively firmly grasp the handle 52 with one hand while maintaining contact on the desired injection site of the ventricular wall. Use of the device 50 can reduce the need/risk for the surgeon/practitioner to remove the depression from the tissue wall 18 and lose sight of the target puncture area when orienting the device 50.

As described above in connection with FIG. 11, the puncture locator device 50 may include an orientation indicator/pointer feature 53 that may be aligned by the surgeon/practitioner with a path/vector to the target anatomy to properly orient the puncture locator device 50. The selected orientation of the device 50 may place the needle trajectory 94 at an approximately 30° angle with respect to a tangent line 95 associated with the ventricle wall 18 at the puncture location. For example, the long axis imaging image (e.g., TEE) 1307 shown in FIG. 13-2 shows a straight line 95 drawn from the left ventricular apex to the approximate introducer/puncture site 159, wherein such line is substantially parallel to the left ventricular wall. Such line may be substantially parallel to and/or co-linear with the tangent line 95 illustrated in image 1306. Furthermore, a line 94 may be drawn from the introducer/puncture site 159 directly to the target tissue anchor deployment site, such as on a target heart valve leaflets (e.g., posterior mitral valve leaflets). The angle θ₂ between these lines can be considered an optimal insertion angle for the introducer/needle puncture. In some implementations, the entry angle θ₂ is between 30 and 45°. In some instances, the angle θ₂ is between 45-90°. The angle θ₂ may be between 30-80°. For example, the angle may be between 70 and 80° in some instances. In some implementations, the surgeon/practitioner may position and orient a tip or other indicator feature 53 of the device 50 in the direction of the target anatomy (e.g., mitral valve).

Additionally or alternatively, certain imaging technology/modality may be implemented to assist in visually orienting the puncture locator device 50. The long-axis echo imaging view of image 1307 may provide an effective side view for viewing the valve leaflets 152, 154, and may provide a view that is up to 90° apart from a bicommissural, as familiar to those having ordinary skill in the are. The cross-sectional long-axis anatomical views images 1306 and 1307 show the left ventricle 3 of the heart 1, as well as the mitral valve 6 and aortic valve 7, which are accessible from the left ventricle 3. The long-axis view of images 1306, 1307 further show the papillary muscles 15 and associated chordae tendinea 16.

At block 1208, the process 1200 involves advancing a needle shaft 48 through a needle channel of the puncture locator device 50 to puncture the wall 18 at the target puncture site 159. Use of the puncture locator device 50 advantageously allows for substantially instant puncture of the heart wall once the target puncture site is determined/located. At block 1210, the process 1200 involves placing an introducer through the puncture channel in the heart wall 18 and using a delivery system to deploy one or more leaflet anchors via access through the introducer 200, as described in detail herein.

### ADDITIONAL EXAMPLES

Provided below is a list of examples, each of which may include aspects of any of the other examples disclosed herein. Furthermore, aspects of any example described above may be implemented in any of the numbered examples provided below.

Example 1: A puncture locator device comprising a handle, a tissue depressor form, and a needle channel that passes through at least a portion of the tissue depressor form.

Example 2: The puncture locator device of any example herein, in particular example 1, wherein the tissue depressor form defines an axis and the needle channel is angled with respect to the axis.

Example 3: The device of any example herein, in particular example 1 or example 2, wherein an axis of the handle is parallel with an axis of the tissue depressor form.

Example 4: The device of any example herein, in particular example 3, wherein the handle is coaxial with the tissue depressor form.

Example 5: The puncture locator device of any example herein, in particular any of examples 1-4, wherein a distal opening of the needle channel is associated with a front side of the tissue depressor form.

Example 6: The puncture locator device of any example herein, in particular any of examples 1-5, wherein the needle channel passes through at least a portion of the handle.

Example 7: The puncture locator device of any example herein, in particular any of examples 1-6, further comprising a stabilizer structure disposed proximal to the tissue depressor form.

Example 8: The puncture locator device of any example herein, in particular example 7, wherein the handle projects proximally from the stabilizer structure and the tissue depressor form projects distally from the stabilizer structure.

Example 9: The puncture locator device of any example herein, in particular example 8, wherein the tissue depressor form emanates and projects from a ventral tissue contact surface of the stabilizer structure.

Example 10: The puncture locator device of any example herein, in particular example 9, wherein the tissue contact surface has a diameter that is greater than a diameter of the tissue depressor form.

Example 11: The puncture locator device of any example herein, in particular any of examples 1-10, further comprising an orientation indicator indicating an orientation of the needle channel.

Example 12: The puncture locator device of any example herein, in particular example 11, wherein the orientation indicator is associated with a stabilizer structure of the puncture locator device.

Example 13: The puncture locator device of any example herein, in particular example 11 or example 12, wherein the orientation indicator comprises a point that is aligned with a plane of the needle channel.

Example 14: The puncture locator device of any example herein, in particular example 13, wherein the point is transverse with respect to an axis of the tissue depressor form.

Example 15: The puncture locator device of any example herein, in particular any of examples 11-14, wherein the orientation indicator has a heart-shaped form.

Example 16: The puncture locator device of any example herein, in particular any of examples 1-15, wherein the puncture locator device is sterilized.

Example 17: A puncture locator device comprising a tissue-contact structure including a stabilizer means and a tissue depressor means, and a needle channel formed in the tissue-contact structure.

Example 18: The puncture locator device of any example herein, in particular example 17, wherein the needle channel has a distal opening on a front surface of the tissue depressor means.

Example 19: The puncture locator device of any example herein, in particular example 17 or example 18, wherein the stabilizer means comprises a heart-shaped form including a point feature that is aligned with a plane of the needle channel.

Example 20: The puncture locator device of any example herein, in particular any of examples 17-20, wherein the stabilizer means comprises a distal tissue-contact surface.

Example 21: The puncture locator device of any example herein, in particular example 20, wherein the tissue depressor form emanates from the tissue-contact surface of the stabilizer means.

Example 22: The puncture locator device of any example herein, in particular any of examples 17-21, wherein the tissue depressor means comprises a bulbous dome that projects from the stabilizer means.

Example 23: The puncture locator device of any example herein, in particular example 22, wherein a diameter of the tissue depressor means expands gradually from an apex of the tissue depressor means to the stabilizer means.

Example 24: The puncture locator device of any example herein, in particular any of examples 17-23, further comprising a manual manipulation means coupled to the stabilizer means.

Example 25: The puncture locator device of any example herein, in particular example 24, wherein the manual manipulation means comprises an elongate handle form.

Example 26: The puncture locator device of any example herein, in particular example 25, wherein the elongate handle form has an elliptical cross-section.

Example 27: The puncture locator device of any example herein, in particular any of example 25 or example 26, wherein the elongate handle form has a rectangular cross-section.

Example 28: The puncture locator device of any example herein, in particular any of examples 25-27, wherein the elongate handle form has an aperture formed therein and the needle channel is accessible through at least a portion of the aperture.

Example 29: The puncture locator device of any example herein, in particular any of examples 17-28, wherein the needle channel has one or more internal longitudinal corrugations.

Example 30: The puncture locator device of any example herein, in particular any of examples 17-29, wherein the puncture locator device is sterilized.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

Depending on the embodiment, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A puncture locator device comprising:
a handle;
a tissue depressor form; and
a needle channel that passes through at least a portion of the tissue depressor form.

2. The puncture locator device of claim 1, wherein:
the tissue depressor form defines an axis; and
the needle channel is angled with respect to the axis.

3. The device of claim 1, wherein an axis of the handle is parallel with an axis of the tissue depressor form.

4. The device of claim 3, wherein the handle is coaxial with the tissue depressor form.

5. The puncture locator device of claim 1, wherein the needle channel passes through at least a portion of the handle.

6. The puncture locator device of claim 1, further comprising a stabilizer structure disposed proximal to the tissue depressor form.

7. The puncture locator device of claim 6, wherein:
the handle projects proximally from the stabilizer structure; and
the tissue depressor form projects distally from a tissue contact surface of the stabilizer structure.

8. The puncture locator device of claim 7, wherein the tissue contact surface has a diameter that is greater than a diameter of the tissue depressor form.

9. The puncture locator device of claim 1, further comprising an orientation indicator indicating an orientation of the needle channel, wherein the orientation indicator is associated with a stabilizer structure of the puncture locator device.

10. The puncture locator device of claim 9, wherein the orientation indicator comprises a point that is aligned with a plane of the needle channel.

11. The puncture locator device of claim 10, wherein:
the orientation indicator has a heart-shaped form; and
the point is formed by an apex of the heart-shaped form.

12. A puncture locator device comprising:
a tissue-contact structure including:
a stabilizer means; and
a tissue depressor means; and
a needle channel formed in the tissue-contact structure.

13. The puncture locator device of claim 12, wherein the needle channel has a distal opening on a front surface of the tissue depressor means.

14. The puncture locator device of claim 12, wherein the stabilizer means comprises a heart-shaped form including a point feature that is aligned with a plane of the needle channel.

15. The puncture locator device of claim 12, wherein:
the stabilizer means comprises a distal tissue-contact surface; and
the tissue depressor form emanates from the tissue-contact surface of the stabilizer means.

16. The puncture locator device of claim 12, wherein the tissue depressor means comprises a bulbous dome that projects from the stabilizer means.

17. The puncture locator device of claim 16, wherein a diameter of the tissue depressor means expands gradually from an apex of the tissue depressor means to the stabilizer means.

18. The puncture locator device of claim 12, further comprising a manual manipulation means coupled to the stabilizer means.
